(19) United States
(12) Patent Application Publication
(10) Pub. No.: US 2024/0298646 A1
(43) Pub. Date: Sep. 12, 2024

(54) ANTIBIOFILM PRESERVATIVE COMPOSITIONS

(71) Applicant: SHARON PERSONAL CARE LTD., Ashdod (IL)

(72) Inventors: Paul SALAMA, Ashdod (IL); Noa ZIKLO, Tel-Aviv-Yafo (IL); Noam STEINMAN, Modi'in Makabim-Re'ut (IL); Inbal TZAFRIR, Holon (IL); Ariel GLICKSBERG, Ashdod (IL)

(21) Appl. No.: 18/280,765

(22) PCT Filed: Mar. 7, 2022
(86) PCT No.: PCT/IL2022/050251

§ 371 Date: Sep. 7, 2023

Related U.S. Application Data
(60) Provisional application No. 63/157,783, filed on Mar. 7, 2021.

(51) Int. Cl. A01N 59/16; A01N 37/06; A01N 37/10; A01N 43/16

(52) U.S. Cl. CPC A01N 59/16; A01N 37/06; A01N 37/10; A01N 43/16

(57) ABSTRACT

Disclosed herein is a composition comprising an antibiofilm agent and a zinc compound in a ratio between 2:1 and 1:5 by weight, respectively. A composition comprising an organic acid and/or a salt thereof and a zinc compound in a ratio between 10:1 and 4:1 by weight, respectively is also disclosed. Formulations and articles comprising the compositions are disclosed. Further methods of inhibiting or reducing the formation of load of a microorganism in and/or on an article and methods of preserving a personal care product are also disclosed.

### CROSS REFERENCE TO RELATED APPLICATIONS

This application claims the benefit of priority of U.S. Provisional Patent Application No. 63/157,783, filed Mar. 7, 2021, the contents of which are all incorporated herein by reference in their entirety.

### FIELD OF THE INVENTION

The present invention is in the field of preservation of personal care formulations.

### BACKGROUND OF THE INVENTION

Wipes used for cosmetic removal, surface cleaning in infants during diaper changes, for wiping ones hands, and the like, often contain compositions formulated for effective cleaning purposes. Disposable wipes are usually saturated with such compositions. Pre-saturated wipes are very convenient for use in numerous applications, such as for cleaning the skin of an infant, child, or adult. These pre-saturated wipes are particularly useful in on-the-go applications, such as in cars or public spaces where traditional cleaning methods, such as soap and water, are not available.

One problem that has become more prevalent in the use of pre-saturated wipes and other similar substrates is the inability to protect the substrate or wipe from microbial contamination. More particularly, even though the substrate is saturated with a wiping composition, many commonly used wiping solutions, including anti-microbial compositions, are not effective against various microorganisms that are known to attack wiping substrates. There is a great need for low cost and effective preservative formulations that can protect the substrate used to apply the wiping composition, especially ones free of specific compounds such as phenoxyethanol, benzyl alcohol, or parabens.

### SUMMARY OF THE INVENTION

In one aspect of the invention, there is provided a composition comprising an organic acid and/or a salt thereof and a zinc compound in a ratio between 10:1 and 4:1 by weight, respectively.

In another aspect of the invention, there is provided a composition comprising an antibiofilm agent and a zinc compound in a ratio between 2:1 and 1:5 by weight, respectively. In some embodiments, the composition further comprises an organic acid and/or a salt thereof.

In some embodiments, the composition comprises between 15% and 95% weight per weight (w/w) of an organic acid and/or a salt thereof.

In some embodiments, the composition comprises between 0.5% and 95% (w/w) of the zinc compound.

In some embodiments, the composition comprises between 1% and 80% (w/w) of the antibiofilm agent.

In some embodiments, the antibiofilm agent comprises a pyrone, salicylic acid or any derivative thereof, or both.

In some embodiments, the pyrone is selected from the group consisting of maltol, ethyl maltol or any derivative thereof.

In some embodiments, the zinc compound comprises zinc acetate, zinc pyrithione, zinc gluconate, or any combination thereof.

In some embodiments, the organic acid is selected from the group consisting of: benzoic acid, dehydroacetic acid, sorbic acid, salicylic acid, lactic acid, citric acid, and any combination thereof.

In some embodiments, the composition comprises between 30% and 90% of the organic acid or salt thereof, between 10% and 25% of the zinc compound and between 5% and 25% of the antibiofilm agent.

In some embodiments, the composition comprises two antibiofilm agents in a 1:1 (w/w) ratio.

In some embodiments, the composition is an antimicrobial composition.

In another aspect of the invention, there is provided a formulation for use in the treatment of a medical, cosmetic and/or cosmeceutical condition comprising less than 2% of the composition of the present invention.

In another aspect of the invention, there is provided an article comprising the composition of the present invention or the formulation of the present invention.

In some embodiments, the article is a personal care product.

In some embodiments, the article is selected from the group consisting of: a fabric, a bandage, a wipe, a pledget, a swab, a suppository, a dressing, a solution, a mousse, a pad, and a patch.

In some embodiments, the product comprises a formulation in the form selected from the group consisting of: a liquid, a solution, a paste, a cream, a lotion, a foam, a gel, an emulsion, an ointment, and a soap.

In some embodiments, the article is for use in the treatment of a condition selected from medical, cosmetic and cosmeceutical condition.

In another aspect of the invention, there is provided a method of inhibiting or reducing the formation of load of a microorganism in and/or on an article, the method comprising contacting the article with the composition of the present invention.

In some embodiments, the microorganism is selected from bacteria, molds and fungi.

In some embodiments, the bacteria are Gram-positive bacteria selected from the group consisting of: Staphylococcus aureus, Bacillus cereus and Staphylococcus epidermidis or Gram-negative bacteria selected from the group consisting of: Escherichia coli and Pseudomonas aeruginosa.

In some embodiments, the fungi is Candida albicans.

In some embodiments, the mold is Aspergillus brasiliensis.

In another aspect of the invention, there is provided a method of preserving a personal care product, comprising adding to personal care product the composition of the present invention.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIGS. 1A-1C include bar graphs of antibiofilm experiments performed without treatment (no treatment) and with treatments with sodium benzoate and potassium sorbate (S.B+P.S), sodium benzoate, potassium sorbate and zinc acetate (S.B+P.S+Z.A), sodium benzoate, potassium sorbate, zinc acetate and maltol as antibiofilm component (S.B+P.S+Z.A+Mal), sodium benzoate, potassium sorbate, zinc acetate, and a mixture of maltol and salicylic acid as antibiofilm component (S.B+P.S+Z.A+Mal+S.A) at different concentrations.

### DETAILED DESCRIPTION OF THE INVENTION

The present invention is directed to a composition comprising an antibiofilm agent and a zinc compound. The present invention is also directed to a composition comprising an organic acid and/or a salt thereof and a zinc compound. The present invention is also directed to a composition comprising an organic acid or salt thereof, a zinc compound and an antibiofilm agent. In some embodiments, the composition is a preservative composition.

The present invention is also directed to formulations comprising the compositions described herein. In some embodiments, the formulation is a personal care formulation.

The present invention is also directed to articles comprising a composition or a formulation described herein. In some embodiments, the article is a wet wipe. In some embodiments, the article is a disposable wet wipe.

The present invention is based, in part, on the finding that compositions (e.g., formulations) according to the present invention, comprising an organic acid and/or a salt thereof and a zinc compound, being at certain predetermined ratios (e.g. synergistically effective amount), exhibit a desired stability as well as improved antimicrobial activities, compared to formulations comprising the same materials but having different volume or weight ratios thereof.

The present invention is based, in part, on the finding that compositions (e.g., formulations) according to the present invention, comprising an antibiofilm agent and a zinc compound, being at certain predetermined ratios, exhibit a desired stability as well as improved antimicrobial activities, compared to formulations comprising the same materials but having different volume or weight ratios thereof.

The present invention is based, in part, on the finding that compositions (e.g., formulations) according to the present invention, comprising an organic acid, an antibiofilm agent and a zinc compound, being at certain predetermined ratios, exhibit a desired stability as well as improved antimicrobial activities, compared to formulations comprising the same materials but having different volume or weight ratios thereof.

In some embodiments, specific ratio (e.g. synergistically effective amount) of the above-mentioned compounds impart the formulation with antimicrobial activities.

The term “antimicrobial” refers to a composition or compound capable of inhibiting the growth of or controlling the growth of microorganisms; antimicrobial compounds include bactericides, bacteristats, fungicides, fungistats, algaecides and algistats.

Herein throughout, by “composition” it is further meant to refer to a formulation.

In some embodiments, the present invention provides non-toxic antimicrobial formulations (also referred to as “blends”).

As used herein, the terms “formulation”, or “blend”, which are used hereinthroughout interchangeably, refer to a vehicle composition in the form of a solution, an emulsion, a lotion, a cream, a gel etc., that optionally further comprises physiologically acceptable carriers and/or excipients and optionally other chemical components such as cosmetically, cosmeceutically or pharmaceutically active agents (e.g., drugs). The formulation can optionally further comprise a carrier, and optionally additional active agents and/or additives e.g., anti-freezing agents).

In some embodiments, the term “by weight” means by weight of the total composition.

### The Composition

A composition comprising an organic acid and/or a salt thereof and a zinc compound in a ratio (e.g. synergistically effective amount) between 10:1 and 4:1, 9:1 and 4:1, 8:1 and 4:1, 7:1 and 4:1, 6:1 and 4:1, 10:1 and 5:1, 9:1 and 5:1, 8:1 and 5:1, 7:1 and 5:1, 6:1 and 5:1, 10:1 and 6:1, 9:1 and 6:1, 8:1 and 6:1, or between 7:1 and 6:1 by weight, respectively, including any range therebetween. Each possibility represents a separate embodiment of the invention. In some embodiments, the composition comprises a single organic acid specie, or a plurality of organic acids (e.g. at least two chemically distinct organic acids), including any salt(s) thereof. In some embodiments, a weight ratio (e.g. synergistically effective amount) between the zinc compound and one or more organic acid(s) is between 1:10 and 1:2, between 1:10 and 1:3, between 1:10 and 1:3.5, between 1:10 and 1:4, between 1:6 and 1:4, between 1:5 and 1:3, between 1:8 and 1:3, between 1:7 and 1:3, between 1:6 and 1:3, between 1:10 and 1:5, between 1:5 and 1:3, including any range between.

According to some embodiments, there is provided a composition comprising between 15% and 95% weight per weight (w/w) of an organic acid and/or a salt thereof and between 5% and 20% (w/w) of a zinc compound.

In some embodiments, the composition of the invention (e.g. antimicrobial composition of the invention) consist essentially of the active ingredients described herein (e.g. (i) a zinc compound and one or more organic acid(s); (ii) antibiofilm agent, a zinc compound and optionally one or more organic acid(s); or (iii) an antibiofilm agent and one or more organic acid(s)).

According to some embodiments, there is provided a composition comprising an antibiofilm agent and a zinc compound. In some embodiments, there is provided a composition comprising an antibiofilm agent and a zinc compound in a ratio (e.g. synergistically effective amount) between 2:1 and 1:5, 1:1 and 1:5, 2:1 and 1:4, 2:1 and 1:3, 2:1 and 1:2, 2:1 and 1:1, 1:1 and 1:5, 1:1 and 1:4, 1:1 and 1:3, or between 1:1 and 1:2, by weight, respectively, including any range therebetween. In some embodiments, a ratio between the antibiofilm agent and the zinc compound within the composition is between 2:1 and 1:2, between 1.5:1 and 1:1.5, or about 1:1, including any range therebetween. Each possibility represents a separate embodiment of the invention. In some embodiments, the composition further comprises an organic acid and/or a salt thereof. In some embodiments, the compositions further comprises between 15% and 95% (w/w) of the organic acid and/or a salt thereof. In some embodiments, a ratio between the organic acid and/or a salt thereof and the zinc compound within the composition is between 1:10 and 1:2, between 1:10 and 1:3, between 1:10 and 1:3.5, between 1:10 and 1:4, between 1:6 and 1:4, between 1:5 and 1:3, between 1:8 and 1:3, between 1:7 and 1:3, between 1:6 and 1:3, between 1:10 and 1:5, between 1:5 and 1:3, including any range between. In some embodiments, a ratio between the organic acid and/or a salt thereof and the zinc compound within the composition is between 1:7 and 1:4 including any range between. In some embodiments, a ratio between the antibiofilm agent and the zinc compound within the composition is between 2:1 and 1:5, 1:1 and 1:5, 2:1 and 1:4, 2:1 and 1:3, 2:1 and 1:2, 2:1 and 1:1, 1:1 and 1:5, 1:1 and 1:4, 1:1 and 1:3, or between 1:1 and 1:2; and a ratio between the organic acid and/or a salt thereof and the zinc compound within the composition is between 1:10 and 1:2, between 1:10 and 1:3, between 1:10 and 1:3.5, between 1:10 and 1:4, between 1:6 and 1:4, between 1:5 and 1:3, between 1:8 and 1:3, between 1:7 and 1:3, between 1:6 and 1:3, between 1:10 and 1:5, between 1:5 and 1:3, including any range between.

According to another embodiments, there is provided a composition comprising an antibiofilm agent and an organic acid. In some embodiments, the composition comprises an antibiofilm agent and a plurality of organic acids (e.g. at least two chemically distinct organic acids). In some embodiments, the plurality of organic acids comprises a first acid and a second acid at a weight ratio (e.g. synergistically effective amount) between 1:3 and 3:1, 1:3 and 1:2, 1:2 and 1:1, 2:1 and 1:2, 2:1 and 1:1, 1:1 and 1:5, including any range therebetween. Each possibility represents a separate embodiment of the invention. In some embodiments, weight ratio between the antibiofilm agent and the plurality of organic acids is between 1:20 and 1:3, between 1:20 and 1:10, between 1:10 and 1:5, between 1:5 and 1:3, including any range between. In some embodiments, the composition is an antimicrobial composition in a form f an aqueous solution or suspension/emulsion/dispersion, wherein the antimicrobial composition comprises an antimicrobial effective amount of the active agents (plurality of organic acids and the antibiofilm agent). In some embodiments, the antimicrobial effective amount comprises less than 5%, less than 3%, less than 2%, less than 1.5%, less than 1% by weight of the total active ingredient content within the antimicrobial composition. In some embodiments, the antimicrobial effective amount comprises between 0.5 and 5%, between 0.5 and 2%, between 0.5 and 1.5%, between 0.5 and 1%, between 0.5 and 3%, between 1 and 3%, between 1 and 2%, between 2 and 5%, between 1 and 1.5%, between 1.5 and 2%, including any range therebetween.

In some embodiments, the composition comprises between 15% and 95% (w/w), 20% and 95% (w/w), 30% and 95% (w/w), 40% and 95% (w/w), 50% and 95% (w/w), 75% and 95% (w/w), 15% and 90% (w/w), 20% and 90% (w/w), 30% and 90% (w/w), 40% and 90% (w/w), 50% and 90% (w/w), 75% and 90% (w/w), 15% and 85% (w/w), 20% and 85% (w/w), 30% and 85% (w/w), 40% and 85% (w/w), 50% and 85% (w/w), 75% and 85% (w/w), 15% and 70% (w/w), 20% and 70% (w/w), 30% and 70% (w/w), 40% and 70% (w/w), or between 50% and 70% (w/w), of an organic acid and/or a salt thereof, including any range therebetween. Each possibility represents a separate embodiment of the invention.

In some embodiments, the compositions comprises between 0.5% and 95%, 0.7% and 95%, 0.9% and 95%, 1% and 95%, 5% and 95%, 10% and 95%, 25% and 95%, 30% and 95%, 50% and 95%, 70% and 95%, 0.5% and 90%, 0.7% and 90%, 0.9% and 90%, 1% and 90%, 5% and 90%, 10% and 90%, 25% and 90%, 30% and 90%, 50% and 90%, 70% and 90%, 0.5% and 75%, 0.7% and 75%, 0.9% and 75%, 1% and 75%, 5% and 75%, 10% and 75%, 25% and 75%, 30% and 75%, 50% and 75%, 0.5% and 55%, 0.7% and 55%, 0.9% and 55%, 1% and 55%, 5% and 55%, 10% and 55%, 25% and 55%, 30% and 55%, 0.5% and 40%, 0.7% and 40%, 0.9% and 40%, 1% and 40%, 5% and 40%, 10% and 40%, or between 25% and 40% weight per weight (w/w) of the zinc compound, including any range therebetween. Each possibility represents a separate embodiment of the invention.

In some embodiments, the composition comprises between 1% and 80% (w/w), 2% and 80% (w/w), 5% and 80% (w/w), 10% and 80% (w/w), 25% and 80% (w/w), 30% and 80% (w/w), 50% and 80% (w/w), 70% and 80% (w/w), 1% and 75% (w/w), 2% and 75% (w/w), 5% and 75% (w/w), 10% and 75% (w/w), 25% and 75% (w/w), 30% and 75% (w/w), 50% and 75% (w/w), 1% and 50% (w/w), 2% and 50% (w/w), 5% and 50% (w/w), 10% and 50% (w/w), 25% and 50% (w/w), or between 30% and 50% (w/w) of the antibiofilm agent, including any range therebetween. Each possibility represents a separate embodiment of the invention.

In some embodiments, any one of the active agents of the invention is or comprises a pharmaceutically-, cosmeceutically, and/or food-acceptable compound.

In some embodiments, the antibiofilm agent comprises a pyrone, salicylic acid, D-Tyrosine, Naringin, Deferiprone, Mannose, Urea, Acetyl Cysteine, Diacetyl Cysteine, Gallic acid, Ethylexylglycerin, Ethylacetoacetate, Hordenine, Tyrosol, Hydroxytyrosol, Tyramine, Lactobionic Acid, Levulinic acid, Anthranilic acid, Methyl anthranilate, Trigonelline, Caffeine, Chlorogenic acid, Tryptophan including any salt thereof, any ester thereof, any derivative thereof, and any combination thereof. In some embodiments, the term “derivative” refers to a stereoisomer (e.g. enantiomer, and/or diastereomer) and/or structural isomer, an ester, a tautomer, and/or any prodrug or precursor of the above disclosed compounds. In some embodiments, the term “derivative” refers to a structural derivative having a similar (or an enhanced) antimicrobial activity. In some embodiments, the pyrone is selected from the group consisting of maltol, ethyl maltol or any derivative thereof.

In some embodiments, the zinc compound comprises Zn(II) cation, and/or a salt thereof. In some embodiments, the zinc compound comprises Zn(II) cation and a counterion, wherein the counterion is any pharmaceutically-, cosmeceutically, and/or food-acceptable counterion. In some embodiments, the zinc compound is or comprises a Zn (II) complex. In some embodiments, the zinc compound comprises zinc acetate, zinc pyrithione, zinc gluconate, zinc lactate, zinc glycinate, zinc chloride, zinc carbonate, zinc phosphate, zinc nitrate, zinc sulfate, or any combination thereof.

In some embodiments, the organic acid comprises between 1 and 7, between 1 and 6, between 2 and 7, between 2 and 6 carbon atoms, including any range between. In some embodiments, the organic acid is or comprises a short chain carboxylic acid. In some embodiments, the organic acid is or comprises a carboxylic acid. In some embodiments, the organic acid is or comprises C1-C7, C1-C6, and/or C2-C7, carboxylic acid.

In some embodiments, the organic acid is selected from the group consisting of: benzoic acid, dehydroacetic acid, sorbic acid, salicylic acid, lactic acid, citric acid, and any combination thereof.

In some embodiments, the composition comprises between 30% and 90% of the organic acid or salt thereof, between 10% and 25% of the zinc compound and between 5% and 25% of the antibiofilm agent. In some embodiments, the composition comprises between 30% and 90% of the organic acid or salt thereof, between 10% and 25% of the zinc compound and between 5% and 25% of a pyrone. In some embodiments, the pyrone is maltol.

In some embodiments, the composition comprises between 30% and 90% of sodium benzoate, between 10% and 25% of zinc acetate and between 5% and 25% of maltol.

In some embodiments, the compositions comprises between 50% and 95% (w/w) of an organic acid and 5% to 20% (w/w) of a zinc compound. In some embodiments, the compositions comprises between 50% and 95% (w/w) of sodium benzoate and 5% to 20% (w/w) of a zinc acetate.

In some embodiments, the composition comprises between 10% and 50% of maltol and between 30% and 95% of zinc acetate.

In some embodiments, the composition comprises two antibiofilm agents in a (w/w) ratio (e.g. synergistically effective amount) of about 1:1, or between about 1.5:1 and 1:1.5. In some embodiments, the composition comprises a pyrone and salicylic acid or any derivative thereof in a (w/w) ratio of about 1:1, or between about 1.5:1 and 1:1.5. In some embodiments, the composition comprises maltol and salicylic acid or any derivative thereof in a (w/w) ratio of about 1:1, or between about 1.5:1 and 1:1.5.

In some embodiments, the composition is an antimicrobial composition. In some embodiments, the composition is a powderous composition. In some embodiments, the composition is a solid composition. In some embodiments, the composition is in a dry state. In some embodiments, the composition is a solid antimicrobial composition.

In some embodiments, the composition of the invention is in a form of a kit. In some embodiments, the constituents are at a synergistically effective amount within the kit, wherein the synergistically effective amount is as described herein. In some embodiments, the kit comprises any one of (I) the organic acid as disclosed herein, and/or a salt thereof and the zinc compound as disclosed herein, in a ratio between 10:1 and 4:1, including any range therebetween (optionally the composition comprising between 15% and 95% weight per weight (w/w) of the organic acid and/or a salt thereof and between 5% and 20% (w/w) of the zinc compound); (II) the antibiofilm agent as disclosed herein, and the zinc compound as disclosed herein, optionally the antibiofilm agent and the zinc compound are in a ratio between 2:1 and 1:5 within the kit, and further optionally comprising between 15% and 95% (w/w) of the organic acid and/or a salt thereof (optionally wherein a ratio between the organic acid and/or a salt thereof and the zinc compound within the kit is between 1:10 and 1:2); and (III) the antibiofilm agent as disclosed herein, and one or more of the organic acid as disclosed herein, wherein a ratio between the antibiofilm agent and one or more of organic acids is between 1:20 and 1:3, between 1:20 and 1:10, between 1:10 and 1:5, between 1:5 and 1:3, including any range between.

In some embodiments, the kit is an antimicrobial or a preservative kit. In some embodiments, the kit comprises one or more compartments (e.g. a first compartment, a second compartment, and optionally a third compartment), wherein each of the compartments comprises one or more of the constituents of the kit, as disclosed herein.

In some embodiments, the kit comprises instructions for mixing of the first compartment, the second compartment, and optionally the third compartment, so as to obtain a composition comprising the constituents of the kit at a predefined ratio (e.g. a synergistically effective amount), as disclosed herein.

In some embodiments, the kit comprises instructions for dilution of the constituents of the kit (e.g. with an appropriate carrier, such as an aqueous solution) in an amount sufficient for obtaining the antimicrobial composition of the invention comprising an antimicrobial effective amount of the constituents. In some embodiments, dilution comprises dispensing a predetermined amount of the kit and adding a predetermined amount of the carrier, so as to obtain a predetermined concentration of the constituents within the antimicrobial composition, wherein the predetermined concentration refers to the antimicrobial effective amount, as described hereinabove.

In some embodiments, the antimicrobial effective amount is as described herein. In some embodiments, the antimicrobial effective amount of the constituents (by total weight of the constituent relative to the weight of the entire antimicrobial composition) is between 0.5 and 5%, including any range between.

In some embodiments, the terms “constituents”, “active ingredients”, and “preservative” including any grammatical from thereof, are used herein interchangeably.

In some embodiments, the kit comprises instructions for dilution and optionally for mixing of the first compartment, the second compartment, so as to obtain the antimicrobial composition of the invention comprising a synergistically effective amount of the constituents. In some embodiments, the kit comprises instructions for dilution and optionally for mixing of the first compartment, the second compartment, and optionally the third compartment, so as to obtain the antimicrobial composition of the invention comprising a synergistically effective amount of the constituents.

In some embodiments, the first compartment, the second compartment and the third compartment of the kit are mixed simultaneously. In some embodiments, the first compartment, the second compartment and the third compartment of the kit are mixed subsequently. In some embodiments, the first compartment, and the second compartment and optionally the third compartment of the kit are applied simultaneously or subsequently. In some embodiments, mixing comprises dosing the compartments in an amount sufficient for obtaining a predetermined molar ratio of the to the active agents within the antimicrobial composition.

In some embodiments, the first compartment, the second compartment and the third compartment of the kit are diluted before mixing.

In some embodiments, dosing comprises dispensing a predetermined amount of the first compartment, a predetermined amount of the second compartment, a predetermined amount of the third compartment and subsequent mixing thereof. In some embodiments, dosing comprises dispensing a predetermined amount of the first compartment, a predetermined amount of the second compartment, so as to obtain a predetermined concentration of the constituents within the antimicrobial composition, wherein the predetermined concentration refers to the antimicrobial effective amount, as described hereinabove.

In some embodiments, the composition is devoid of phenoxyethanol. In some embodiments, the composition is devoid of benzyl alcohol. In some embodiments, the composition is devoid of parabens.

As demonstrated in the Examples section that follows, the present inventors have shown that compositions (e.g., formulations) comprising an antibiofilm agent and a zinc compound, being at certain predetermined ratios, exhibit a desired stability as well as improved antimicrobial activities, compared to other compositions comprising different materials from the invented formulations or from formulations comprising the same materials but having different volume or weight ratios thereof.

As demonstrated in the Examples section that follows, the present inventors have shown that compositions (e.g., formulations) comprising an organic acid, an antibiofilm agent and a zinc compound, being at certain predetermined ratios, exhibit a desired stability as well as improved antimicrobial activities, compared to other compositions comprising different materials from the invented formulations or from formulations comprising the same materials but having different volume or weight ratios thereof.

As demonstrated in the Examples section that follows, the present inventors have shown that a specific ratio of the above-mentioned compounds impart the formulation with antimicrobial activities.

The present inventors have also shown that the disclosed formulation can be used to impart to articles the antimicrobial activities.

### The Formulation

According to some embodiments of the present invention there is provided a formulation comprising less than 2% of the composition as described herein. In some embodiments, the formulation is antimicrobial formulation comprising an acceptable carrier and an antimicrobial effective amount of any one of the compositions disclosed herein. In some embodiments, the formulation is antimicrobial formulation formulated for use in an article susceptible to microbial growth as a preservative. In some embodiments, the article is selected from a cosmeceutical article, a pharmaceutical article, and/or a food-grade article. In some embodiments, the formulation is selected from a solid formulation, a liquid formulation (e.g. an aqueous formulation), a gel, an emulsion, a cream, a foam, or any combination thereof.

In some embodiments, the antimicrobial effective amount comprises less than 5%, less than 3%, less than 2%, less than 1.5%, less than 1% by weight of the composition of the invention, relative to the total weight of the formulation. In some embodiments, the antimicrobial effective amount comprises less than 5%, less than 3%, less than 2%, less than 1.5%, less than 1% by weight of the total active ingredient content within the antimicrobial formulation. In some embodiments, the antimicrobial effective amount comprises between 0.5 and 5%, between 0.5 and 2%, between 0.5 and 1.5%, between 0.5 and 1%, between 0.5 and 3%, between 1 and 3%, between 1 and 2%, between 2 and 5%, between 1 and 1.5%, between 1.5 and 2% of the total active ingredient content including any range therebetween.

In some embodiments, the formulation is for use in a cosmeceutical article. In some embodiments, the formulation is for use in a personal care article. In some embodiments, the formulation is for use in pharmaceutical composition or a medical device. In some embodiments, the formulation is a personal care formulation.

In some embodiments, the acceptable carrier is selected from a pharmaceutically acceptable carrier, a cosmeceutically acceptable carrier, and a food acceptable carrier. In some embodiments, the acceptable carrier comprises a surfactant. In some embodiments, the acceptable carrier comprises an aqueous solvent and a surfactant.

In some embodiments, a w/w concentration of the acceptable carrier within the formulation is between 50 and 99%, between 60 and 99%, between 70 and 99%, between 80 and 99%, between 90 and 99%, between 50 and 70%, between 70 and 90%, including any range therebetween. In some embodiments, the formulation further comprises an additive, such as thickener, fragrance, a buffering agent, a stabilizer, an antioxidant, etc.

In some embodiments, a w/w concentration of the surfactant within the formulation is between 0.1 and 10%, between 0.1 and 0.5%, between 0.5 and 10%, between 1 and 10%, between 0.5 and 1%, between 1 and 3%, between 3 and 5%, between 5 and 10%, including any range therebetween.

In some embodiments, the surfactant is selected from an anionic surfactant, a cationic surfactant, and a non-ionic surfactant, including any combination thereof.

In some embodiments, the formulation is an anionic formulation, cationic formulation, zwitterionic formulation, or nonionic formulation.

Non-limiting examples of surfactants include but are not limited to: non-ionic surfactants (e.g., glyceryl monolinoleate glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically, cosmeceutically and/or food acceptable salts of fatty acids such as stearic, oleic, palmitic, and lauric acids), cationic surfactants (e.g. pharmaceutically, cosmeceutically and/or food acceptable quaternary ammonium salts such as benzalkonium chloride, benzethonium chloride, and cetylpyridinium chloride) or any combination thereof. Additional non-limiting examples of anionic surfactants include but are not limited to: (C₆-C₈)alkyl-sulfate and/or sulfonate (e.g., sodium or potassium lauryl sulfate, sodium or potassium dodecyl sulfate), fatty alcohol ether sulfate salt (e.g., (C₁₂-C₁₄)alkyl-O—(CH₂CH₂O)₂—SO₃, ZOHARPON ETA 27), polyacrylate (e.g., sodium or potassium polyacrylates), or any combination thereof. Additional non-limiting examples of non-ionic surfactants include but are not limited to: alkyl-polyglycoside (e.g., Triton CG 110, APG 810), polyethyleneglycol-(C₁₁-C₁₅)alkyl-ether (such as Imbentin AGS/35), alkoxylated fatty alcohol (such as Plurafac LF221), or any combination thereof. In some embodiments, the nonionic surfactant is or comprises Decyl glucoside. In some embodiments, the cationic surfactant is or comprises guar hydroxypropyl trimonium chloride. In some embodiments, the anionic surfactant is or comprises Disodium Cocoamphodiacetate.

In some embodiments, a cationic formulation comprises 5%, less than 4%, less than 3%, less than 2%, or less than 1%, by weight, of a composition as described herein, including any value therebetween. Each possibility represents a separate embodiment of the invention.

In some embodiments, an anionic formulation comprises 5%, less than 4%, less than 3%, less than 2%, or less than 1%, by weight, of a composition as described herein, including any value therebetween. Each possibility represents a separate embodiment of the invention.

In some embodiments, a nonionic formulation comprises 5%, less than 4%, less than 3%, less than 2%, or less than 1%, by weight, of a composition as described herein, including any value therebetween. Each possibility represents a separate embodiment of the invention.

In some embodiments, the formulation is a personal care composition.

In some embodiments, the formulation is not pH dependent.

In some embodiments of the present invention, the formulation further comprises a buffer solution or a pH adjuster to control the desired pH of the formulation.

The formulation of the invention can be prepared by any commonly used method for preparing a composition of materials. For example, the components of the formulations may be added and mixed together, or one of the components may be added to the other in the form of a solution which may, if desired, be evaporated or lyophilized after mixing for obtaining a homogeneous and stable solution or suspension.

As used herein, the term “physiologically acceptable” means approved by a regulatory agency of the Federal or a state government or listed in the U.S. Pharmacopeia or other generally recognized pharmacopeia for use in animals, and more particularly in humans.

As used herein, the term “excipient” refers to an inert substance added to a formulation as described herein to further facilitate processes and administration of the active ingredients.

In some embodiments, the disclosed composition, in any embodiment thereof, is a stable formulation.

As used herein the terms “stable formulation”, or “long-lasting formulation”, mean that the formulation remains in a state or condition of sufficient stability to have utility as a personal care agent, while maintaining the antimicrobial activity (with ±20% variation). For example, and without limitation, the formulation has a sufficient stability to allow storage at a convenient temperature, e.g., between 10° C. and 30° C., under ambient atmosphere (normal atmospheric pressure, atmospheric gases, etc.) for a reasonable period of time of e.g., longer than one month, longer than three months, longer than six months, and longer than one year or between 1 week and 1 y, between 1 w and 2 y, including any range between. In some embodiments,

In some embodiments, the disclosed composition is incorporated into a substrate (such as a textile substrate, a moist textile substrate, a moist substrate, an edible matter, a plastic substrate, and/or a formulation such as a solid formulation, a liquid formulation, a gel, an emulsion, a foam, or any combination thereof) susceptible to microbial growth as a preservative (or an antimicrobial composition).

In some embodiments, the disclosed composition is incorporated in a preservative or in a personal care system in a concentration (by total weight) of e.g., 0.1%, 0.5%, 1%, 1.5%, 2%, 2.5%, 3%, 3.5%, 4%, 4.5%, 5%, 5.5%, 6%, 6.5%, 7%, 6.5%, 8%, 8.5%, 9%, 9.5%, 10%, 10.5%, 11%, 11.5%, 12%, 12.5%, 13%, 13.5%, 14%, 14.5%, or 15%, including any value and range therebetween. Each possibility represents a separate embodiment of the invention.

As used herein, the phrase “personal care” refers to compositions that can be formulated in various cosmetic and pharmaceutical consumer products utilizing a variety of delivery systems and carrier bases. Such consumer product forms include, but are not limited to, fabric, a bandage, a wipe, a baby wipe, a pledget, a swab, a suppository, shampoos, aftershaves, sunscreens, body and hand lotions, skin creams, liquid soaps, bar soaps, bath oil bars, shaving creams, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, conditioners, hair lighteners, coloring and non-coloring hair rinses, hair grooming aids, hair tonics, spritzes, styling waxes, band-aids, and balms.

### Articles Comprising the Formulations

According to an aspect of some embodiments of the present invention there is provided an article which comprises any one of the formulations described herein.

According to an aspect of some embodiments of the present invention there is provided an article which comprises any one of the compositions described herein.

In some embodiments, the article is selected from a cosmeceutical article (e.g. a cream, ointment, foam, solution, lotion, gel, shampoo, soap, show gel, cleansing solution, etc.) and a personal care product.

Some embodiments of this aspect of present embodiments are included hereinabove, under “Composition” or “The Formulation”, and form an integral part of embodiments relating to “Articles comprising the Formulations”.

According to an aspect of some embodiments of the present invention, there is provided a pharmaceutical, cosmetic or cosmeceutical product comprising the formulation described in any of their respective embodiments herein, for use in treating a medical, cosmetic or cosmeceutic condition, as described herein.

According to an aspect of some embodiments of the present invention, the formulation described in any of their respective embodiments herein is used as, or a part of, a preservative in any pharmaceutical, cosmetic or cosmeceutical product or in any article as describe herein.

As used herein, “preservative” is used to prevent the growth of bacteria, fungi and/or molds in any personal care composition or formulation.

According to a further aspect of some embodiments of the present invention, there is provided a use of the formulation described herein in the manufacture of a pharmaceutical, cosmetic or cosmeceutical product, which can be used in treating a medical, cosmetic or cosmeceutic condition, as described herein.

In some embodiments, there is provided a method of treating a medical, cosmeceutical or cosmetic condition treatable by a topical or transdermal administration, the method comprising topically applying the formulation described herein (e.g., in the context of a pharmaceutical, cosmetic or cosmeceutic product) to a skin or mucosal tissue of a subject afflicted by the condition.

The phrases “topical” “topical administrations” and or any grammatical derivative thereof, is meant to encompass applications, which include, without limitation, dermal applications, ophthalmic application, vaginal application, rectal application and intranasal application.

Medical, cosmetic or cosmeceutical conditions that can benefit from containing the formulations described herein when applied topically, with or without an additional active ingredient, include, but are not limited to, infections caused by pathogenic microorganisms, as discussed in further detail hereinbelow, wounds, particularly when associated with an infection, acne, skin infections, viral blisters such as one caused by herpes, sexual dysfunction such as erectile dysfunction.

Hence, according to some embodiments of the present invention, the pharmaceutical, cosmetic or cosmeceutical formulation or product further comprises an antimicrobial agent, as an additional pharmaceutically active agent.

Microbial infections include any infection caused by a pathogenic microorganism, including, bacterial infection, fungal infection, protozoal infection, viral infection and the like, e.g., molluscum contagiosum (a viral infection of the skin or occasionally of the mucous membranes), fungal nail infections, and cutaneous leishmaniasis.

Topical bodily sites include skin, mucosal tissue, eye, ear, nose, mouth, rectum and vagina.

In some embodiments, there is provided an article (e.g., a medical device such as a bandage or adhesive patch), a formulation, or a product, as described herein, configured for topical application, whereby a condition treatable by such as article, product or formulation is an infection caused by a microorganism.

In some embodiments of the present invention, the article is e.g., a fabric, a bandage, a wipe (e.g., a wet wipe), a pledget, a swab, a suppository, a dressing, a solution, a mousse, a pad, or a patch.

In some exemplary embodiments of the present invention, the article is in the form of a solution, a paste, a cream, a lotion, a foam, a gel, an emulsion, an ointment, or a soap.

In some embodiments, the personal care formulation of the present invention can be used to treat skin tissue or on damaged or unhealthy skin tissue.

The phrase “damaged or unhealthy skin tissue” as used herein refers to a deviation from healthy functional skin tissue. In the case of skin-a skin that is weaker, less elastic, and is more prone to injury than healthy skin. The structure of unhealthy or damaged skin is inferior to that of healthy skin (for example, the dermis and epidermis contain fewer cells and collagen).

The phrase “healthy skin tissue” as used herein refers to skin that is strong, elastic, smooth and plump. One purpose of treating healthy skin is to prevent deterioration of skin induced by aging or environmental stress including, but not limited to, microbial infection.

The term “damaged” as used herein, or any grammatical derivative thereof, refers broadly to injuries to the skin and subcutaneous tissue as well as internal organs initiated in any one of a variety of ways (e.g., pressure sores from extended bed rest, wounds induced by trauma, wounds received during or following a surgical procedure and the like) and with varying characteristics. Examples include, but are not limited to, bruises, scrapes, burn wounds, sunburn wounds, incisional wounds, excisional wounds, surgical wounds, necrotizing fascitis, ulcers, venous stasis ulcers, diabetic ulcers, decubitus ulcers, aphthous ulcers, pressure ulcers, scars, alopecia areata, dermatitis, allergic contact dermatitis, atopic dermatitis, berloque dermatitis, diaper dermatitis, dyshidrotic dermatitis, psoriasis, eczema, erythema, warts, anal warts, angioma, cherry angioma, athlete's foot, atypical moles, basal cell carcinoma, Bateman's purpura, bullous pemphigoid, Candida, chondrodermatitis helicis, Clark's nevus, cold sores, condylomata, cysts, Darier's disease, dermatofibroma, Discoid Lupus Erythematosus, nummular eczema, atopic eczema, dyshidrotic eczema, hand eczema, Multiforme Erythema Nodosum, Fordyce's Condition, Folliculitis Keloidalis Nuchae, Folliculitis, Granuloma Annulare, Grover's Disease, heat rash, herpes simplex, herpes zoster (shingles), Hidradenitis Suppurativa, Hives, Hyperhidrosis, Ichthyosis, Impetigo, Keratosis Pilaris, Keloids, Keratoacanthoma, Lichen Planus, Lichen Planus Like Keratosis, Lichen Simplex Chronicus, Lichen Sclerosus, Lymphomatoid Papulosis, Lupus of the Skin, Lyme Disease, Lichen Striatus, Myxoid Cysts, Mycosis Fungoides, Molluscum Contagiosum, Moles, Nail Fungus, Necrobiosis Lipoidica Diabeticorum, Nummular Dermatitis, Onychoschizia, Onychomycosis, Pityriasis Lichenoides, Pityriasis Rosea, Pityriasis Rubra Pilaris, Plantar Warts, Poison Ivy, Poison Oak, Pompholyx, Pseudofolliculitis Barbae, Pruritus Ani and Pityriasis Alba.

### Antimicrobial Activity

According to an aspect of some embodiments of the present invention there is provided a method of inhibiting or reducing or retarding the formation of load of a microorganism and/or the formation of a biofilm, in and/or on an article.

In some embodiments, the method comprises contacting the article with any one of the formulations disclosed herein. In some embodiments, the method comprises contacting the article with any one of the compositions disclosed herein.

In some embodiments, the method comprises incorporating in and/or on the article any one of the compositions disclosed herein, including any of the respective embodiments thereof. In some embodiments, the method comprises incorporating in and/or on the article any one of the formulations disclosed herein, including any of the respective embodiments thereof. The article can be any one of the articles described herein.

According to an aspect of some embodiments of the present invention there is provided a method of preserving a cosmetic product, comprising adding to the cosmetic product any one of the compositions disclosed herein.

According to an aspect of some embodiments of the present invention there is provided a method of preserving a personal care product, comprising adding to the personal care product any one of the compositions disclosed herein.

In some embodiments, the components in the formulation act in synergism.

In some embodiments, the term synergism, or any grammatical derivative thereof, is defined as the simultaneous action of two or more compounds in which the total response of an organism to the combination is greater than the sum of the individual components. Although many combinations of antimicrobial compounds have been studied, a synergistic effect is rarely revealed and the global use of antimicrobial combinations with synergistically enhanced activity is rather limited.

Herein “antimicrobial activity” is referred to as an ability to inhibit (prevent), reduce or retard bacterial growth, fungal growth, biofilm formation or eradicate living bacterial cells, or their spores, or fungal cells or viruses in a suspension or in a moist environment.

Herein, inhibiting or reducing or retarding the formation of load of a microorganism refers to inhibiting, reducing, or retarding growth of microorganisms and/or eradicating a portion or all of an existing population of microorganisms. Thus, formulations described herein can be used both in reducing the formation of microorganisms on or in an article, and in killing microorganisms in or on an article or a living tissue.

The microorganism can be, for example, a unicellular microorganism (prokaryotes, archaea, bacteria, eukaryotes, protists, fungi, algae, molds, yeast, euglena, protozoan, dinoflagellates, apicomplexa, trypanosomes, amoebae and the likes), or a multicellular microorganism.

In some embodiments, the microorganism comprises bacterial cells of bacteria such as, for example, Gram-positive and Gram-negative bacteria.

In some embodiments, the Gram-positive bacteria are Staphylococcus aureus, Staphylococcus epidermidis, and Bacillus cereus.

In some embodiments, the Gram-negative bacteria are Escherichia coli, Pseudomonas aeruginosa, and Burkholderia cepacia.

In some embodiments of the present invention, the fungi is Candida albicans.

In some embodiments, the mold is Aspergillus brasiliensis.

The term “biofilm”, as used herein, refers to an aggregate of living cells which are stuck to each other and/or immobilized onto a surface as colonies. The cells are frequently embedded within a self-secreted matrix of extracellular polymeric substance (EPS), also referred to as “slime”, which is a polymeric sticky mixture of nucleic acids, proteins and polysaccharides.

In the context of the present embodiments, the living cells forming a biofilm can be cells of a unicellular microorganism (prokaryotes, archaea, bacteria, eukaryotes, protists, fungi, algae, euglena, protozoan, dinoflagellates, apicomplexa, trypanosomes, amoebae and the likes), or cells of multicellular organisms in which case the biofilm can be regarded as a colony of cells (like in the case of the unicellular organisms) or as a lower form of a tissue.

In the context of the present embodiments, the cells are of microorganism origins, and the biofilm is a biofilm of microorganisms, such as bacteria and fungi. The cells of a microorganism growing in a biofilm may be physiologically distinct from cells in the “planktonic form” of the same organism, which by contrast, are single-cells that may float or swim in a liquid medium. Biofilms can go through several life-cycle steps which include initial attachment, irreversible attachment, one or more maturation stages, and dispersion.

The term “antibiofilm” refers to the capacity of a substance to disturb the formation of a biofilm of bacterial, fungal and/or other cells, and/or to affect a reduction in the rate of buildup of a biofilm of bacterial, fungal and/or other cells, on a surface of a substrate.

As used herein, the term “preventing” in the context of antimicrobial, indicates that the growth rate of the microorganism cells is essentially nullified or is reduced by at least 20%, at least 30%, at least 40%, at least 50%, at least 60%, at least 70%, at least 80%, at least 90%, including any value therebetween, of the appearance of the microorganism in a comparable situation lacking the presence of the antimicrobial formulation of the invention or an article containing same. Each possibility represents a separate embodiment of the invention. Alternatively, preventing means a reduction to at least 15%, 10%, or 5% of the appearance of the microorganism cells in a comparable situation lacking the presence of the formulation or an article containing same. As used herein, the term “reducing” in the context of antimicrobial, indicates that the growth rate (and or microbial loading expressed in CFU or CFU/ml) of the microorganism (including spores, cells, or biofilm thereof) is essentially reduced as compared to a similar article devoid of the antimicrobial formulation or composition of the invention. In some embodiments, the term “essentially reduced” comprises at least 2 times, at least 5 times, at least 10 times, at least 50 times, at least 100 times, at least 1000 times, at least 10.000 times, at least 100.000 times, at least 1000.000 times CFU reduction as compared to the CFU content of a similar article devoid of the antimicrobial formulation or composition of the invention.

Methods for determining a level of appearance of a microorganism cells are known in the art.

Such articles take advantage of the improved antimicrobial activity exhibited by the formulations as described herein.

An article, according to these embodiments, can be also a living tissue, for example, a skin or mucosal tissue, as described herein.

In the context of the present embodiments, the formulations, articles and methods described herein may be used to produce cell inhibiting surface, or a microbial cell killing surface, that remains active for extended periods. Such an antimicrobial surface may not need an additional treatment with antimicrobial compositions, clean-up treatments to effect decontamination and cosmetic painting, thereby simplifying upkeep of the physical condition and appearance of microbial infestation prone surfaces. It is contemplated that in some embodiments, the formulations of the present invention may be easily applied to susceptible surfaces in advance of and/or during exposure to a microbial organism.

### General

As used herein the term “about” refers to +10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

### Protocol Antibiofilm Assay in 96-Well Microtiter Plate

The assay quantifies the dye bound to biofilm. It quantifies all biomass (live, dead and also matrix of biofilm).

### Preparation of Bacterial Cultures

1 bead from P. aeruginosa stock was taken in −80° C. and placed on a pre-heated TSA and incubated overnight. O.D. was adjusted to 0.3 in saline (equal to ˜10⁸ cfu/mL) then diluted 1/100 ml MH (Final dilution inoculate the plate receive ˜5×10⁵ cfu/mL).

### Procedure

200 ul of antibiofilm agent were added to the first well. Aliquot 100 μL of cell suspension (P. aeruginosa O.D=0.3, 1/100 MH) into 96-well microtiter plates (After serial dilution of the antibiofilm). A blank well containing uninoculated medium was used as a control. The plate was incubate for 20-24 hr at 32° C. with shaking at 85 rpm. Planktonic cells were gently removed by turning the plate down above waste container then washed 2 times with deionized water. Biofilms were fixed by heating at 60° C. for 1 hr and then cooled to room temperature. 250 μL 0.1% Crystal violet (in water) solution was added to wells. The liquid was removed and the plate submerged into container with deionized water. The rinsing was repeated three times. The plate was dried overnight (plate face down in operating chemical hood), until all excess water was evaporated, prior to proceeding with quantitation. 260 μL (130 μL×2) 30% acetic acid (in water) was added to each well. After 10 minutes at room temperature, 125 μL of each sample was transferred to a 96-well optically clear plate, and the optical density of all samples in plate was measured at 570 nm. Microorganisms Strains and Growth Conditions

Escherichia coli (ATCC 8739), Staphylococcus aureus (ATCC 6538), P. aeruginosa (ATCC 9027), Candida albicans (ATCC 10231), and Aspergillus brasiliensis (ATCC 16404) were obtained from ATCC and cultured according to the manufacturer's instructions. These microorganisms are routinely used for minimum inhibitory concentration (MIC), checkerboards and challenge tests according to the US and EU recommendations for the efficacy of preservatives in the cosmetic industry. Bacterial strains were maintained in tryptic soy agar (TSA) (Hy-labs, Rehovot, Israel), while yeast and mold strains were maintained in sabouraud dextrose agar (SDA) (Hy-labs, Israel), supplemented with oxytetracycline at a concentration of 1% (Hy-labs, Israel). Preservative efficacy assays (challenge tests) were performed in order to enumerate the number of microorganisms present in a formulation containing the preservative system, while the diluent solution was supplemented with neutralizers containing 4% Tween 20 and 0.5% lecithin (Hy-labs, Israel).

### Minimum Inhibitory Concentration (MIC) and Checkerboard Assay

The minimum inhibitory concentration (MIC in 96 well plates) of sodium benzoate, potassium sorbate, zinc acetate, maltol and salicylic acid were evaluated using a microdilution broth assay against the five pharmacopeia strains. Briefly, growth of E. coli, S. aureus, P. aeruginosa, P. gergoviea, B. cepacia, C. albicans, and A. brasiliensis was observed during incubation with two-fold serial dilutions of the abovementioned compounds in Mueller Hinton (MH) broth, using a 96-well plate (BIOFUL, Israel). For the checkerboard assay using a 96-well plate, decreasing concentrations of one compound were added from top to bottom of the plate using serial dilutions. Decreasing concentrations of the second compound were added to the plate from the right, having the highest concentration, to the left side of the plate with the lowest concentration. In both MIC and checkerboard assays, wells were inoculated with 100 μl of test cultures with a final inoculum of 5×10⁵ cfu/mL of bacteria and 5×10³ cfu/mL of fungi. Bacteria inoculated plates were incubated for 24 h at 32° C. with shaking, while fungi inoculated plates were incubated at 23° C. for 48-72 h with shaking. Microorganism growth was evaluated by a spectrophotometer O.D. reads (BioTek, Israel).

### MIC Method in Test Tubes

In additional exemplary procedure, MIC evaluation test with Candida albicans was performed in test tubes. Growth in this case is visually evaluated. A pure culture of a single microorganism was grown in Tryptic soy broth, or Sabouraud dextrose broth for yeast and mold.

In another exemplary procedure, MIC evaluation test with A. brasiliensis was performed in test tubes. Growth in this case is visually evaluated. A pure culture of a single microorganism was grown in Tryptic soy broth, or Sabouraud broth for yeast and mold.

The culture is standardized using standard microbiological techniques to have a concentration of very near 1 million cells per milliliter.

The antimicrobial agent is diluted a number of times, with Tryptic soy broth or Sabouraud broth for fungi. After the antimicrobial agent had been diluted, a volume of the standardized inoculums was added to each dilution vessel, bringing the microbial concentration to approximately 1,000,000 cells per milliliter. The inoculated, serially diluted antimicrobial agent was incubated at an appropriate temperature for the test organism, 24 hours for bacteria 48 for yeast and 72 for mold.

After incubation, the series of dilution vessels was observed for microbial growth, usually indicated by turbidity and/or a pellet of microorganisms in the bottom of the vessel. The last tube in the dilution series that does not demonstrate growth corresponds with the MIC of the antimicrobial agent.

The microorganism strains used for the test were E. coli ATCC 8739, P. aeruginosa ATCC 9027, S. aureus ATCC 6538, C. albicans ATCC 10231, A. brasiliensis ATCC 16404.

### Synergistic Effect

Measuring synergy between preservatives can be done by the widely described Fractional Inhibitory Concentration Index (FICI). While the MIC is defined as the lowest concentration of molecule alone or in combination that inhibited visible growth, the in vitro interactions can be quantified as FICI and calculated using the following formula:

$$FICI = \frac{MIC of compound {A}_{combined}}{MIC of compound {A}_{alone}} + \frac{MIC of compound {B}_{combined}}{MIC of compound {B}_{alone}}$$

FICI values were interpreted as follows: <0.5 synergy, 0.5-0.75 partial synergy, 0.75-1.0 additive effect 1.0-4.0 no interaction, and a FICI of >4.0 was defined as antagonism.

### Challenge Test

Challenge tests of preservative efficacy in formulations were performed according to the ISO 11930 regulations. The total count for any prior contamination was performed for all formulations. Specifically, in order to evaluate the antimicrobial and antifungal activity of the preservative system in wet wipes, samples were inoculated separately with each microorganism at a final concentration of 106 cfu/mL for bacteria and 105 cfu/mL for yeast and mold. Samples were incubated in the dark at 22° C. for 28 days. The preservative efficacy was determined by sampling 1 g from the formulation at each time-point of 2, 7, 14, 21, and 28 days. To enumerate the microorganisms at each time point, serial dilutions were made up to 10-4, and 1 mL were seeded in duplicates onto a petri dish with the appropriate media TSA/SDA (bacteria vs. yeast and mold, respectively), using the pour plate method. Plates were incubated at 32° C. for three days for bacteria while yeast and mold were incubated at 22° C. for five days until the enumeration of viable microorganisms.

The nonionic and anionic wet wipes formulations are shown in Table 1.

TABLE 1

| | |
|---|---|
| Nonionic | Anionic |

| | | | |
|---|---|---|---|
| Water | q.s. | Water | q.s. |
| Chamomile extract | 0.05% | Chamomile extract | 0.05% |
| Aloe Vera extract | 0.001% | Aloe Vera extract | 0.001% |
| D-Panthenol | 0.05% | D-Panthenol | 0.05% |
| Glycerin | 0.5% | Glycerin | 0.5% |
| Citric acid | q.s. | Citric acid | q.s. |
| Decyl glucoside | 0.8% | Polysorbate 20 | 0.6% |
| Preservative | 0.5-1% | Disodium | 0.2% |
| | | Cocoamphodiacetate | |
| | | Preservative | 0.5-1% |
| pH | 5.5 | pH | 5.5 |

### Example 1

### Antibiofilm Properties

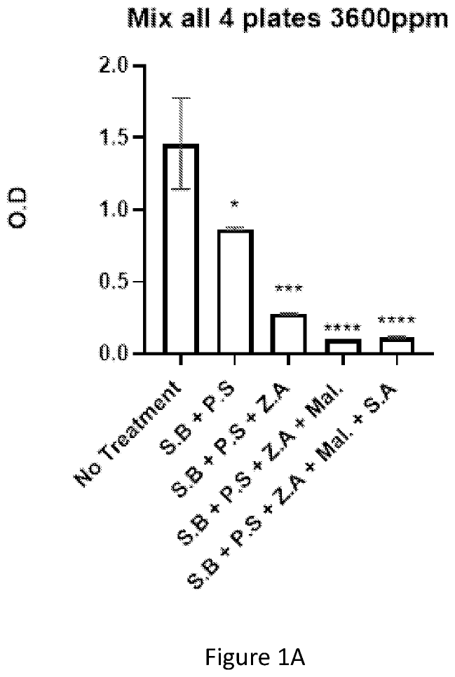

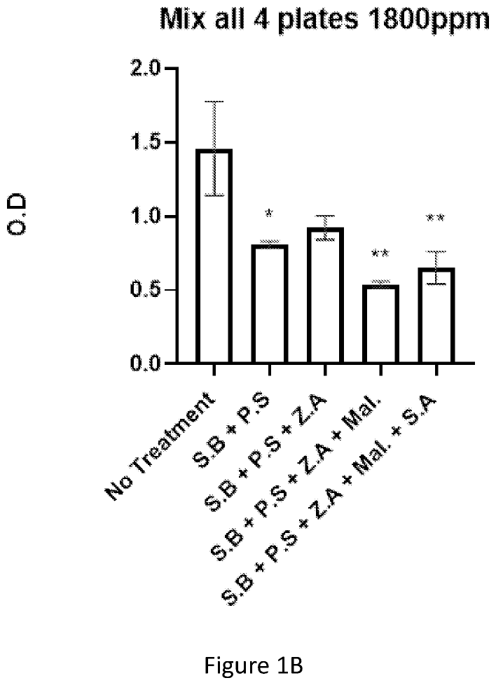

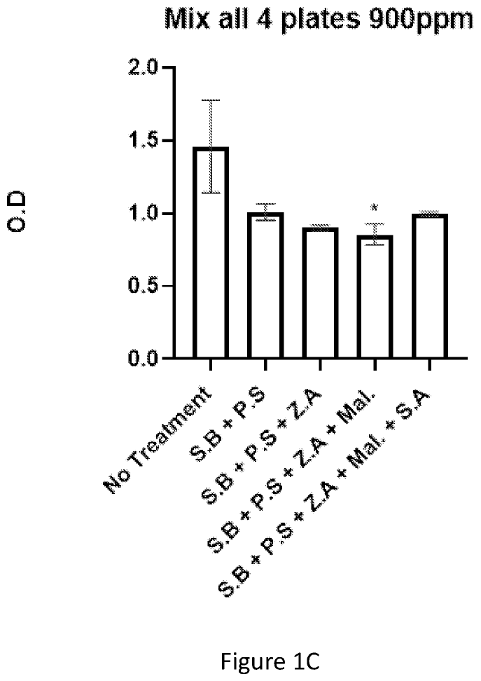

FIGS. 1A-1C and Table 2 present the results of experiments performed without biofilm agent (no treatment) and with sodium benzoate and potassium sorbate (S.B+P.S), sodium benzoate, potassium sorbate and zinc acetate (S.B+P.S+Z.A), sodium benzoate, potassium sorbate, zinc acetate and maltol (S.B+P.S+Z.A+Mal), sodium benzoate, potassium sorbate, zinc acetate, maltol and salicylic acid (S.B+P.S+Z.A+Mal+S.A) at different concentrations.

TABLE 2

| | | | | S.B + P.S + | S.B + P.S + Z.A + |
|---|---|---|---|---|---|
| | | S.B + P.S | S.B + P.S + Z.A | Z.A + Mal. | Mal. + S.A |
| 3600 | No | (2560:1040 | (2140:880:580 | (1850:750:500:500 | (1850:750:500:250:250 |
| ppm | treatment | ppm) | ppm) | ppm) | ppm) |
| Mean | 1.5 | 0.87 | 0.28 | 0.099 | 0.12 |
| Std. | 0.32 | 0.012 | 0.0048 | 0.00018 | 0.0069 |
| Deviation | | | | | |
| Reduction | | −42% | −81.3% | −93.4% | −92% |

| | | | | S.B + P.S + | S.B + P.S + Z.A + |
|---|---|---|---|---|---|
| | | S.B + P.S | S.B + P.S + Z.A | Z.A + Mal. | Mal. + S.A |
| 1800 | No | (1280:520 | (1070:440:290 | (925:375:250:250 | (925:375:250:125:125 |
| ppm | treatment | ppm) | ppm) | ppm) | ppm) |
| Mean | 1.5 | 0.81 | 0.92 | 0.54 | 0.65 |
| Std. | 0.32 | 0.025 | 0.081 | 0.023 | 0.11 |
| Deviation | | | | | |
| Reduction | | −46% | −38.7% | −64% | −56.7% |

| | | | | S.B + P.S + | S.B + P.S + Z.A + |
|---|---|---|---|---|---|
| | | S.B + P.S | S.B + P.S + Z.A | Z.A + Mal. | Mal. + S.A |
| 900 | No | (640:260 | (535:220:145 | (462.5:187.5:125:125 | (462.5:187.5:125:62.5:62.5 |
| ppm | treatment | ppm) | ppm) | ppm) | ppm) |
| Mean | 1.5 | 1.0 | 0.90 | 0.85 | 1.0 |
| Std. | 0.32 | 0.057 | 0.018 | 0.073 | 0.013 |
| Deviation | | | | | |
| Reduction | | −33.3% | −40% | −43.3% | −33.3% |

### Example 2

### Anti Microbial Activity Evaluation

Table 3 presents the antimicrobial activity results (MIC; relative efficacy) of different preservatives and fractional inhibitory concentration index (FICI) of ethylhexylglycerin (EHG), zinc acetate and maltol.

TABLE 3

| Preservative | E. coli | S. aureus | P. aeruginosa | C. albicans | A. brasiliensis |
|---|---|---|---|---|---|
| Zinc Acetate | 312 | 160 | 312 | 110 | 100 |
| EHG | 5000 | 2500 | >5000 | 2500 | 1250 |
| Maltol | 5000 | 5000 | 1250 | 1875 | 1250 |
| EHG 50%, Zinc | 1500 | 1500 | 3000 | 375 | 188 |
| Acetate 33.33%, | | | | | |
| Maltol 16.66% | | | | | |
| FICI | 1.8 | 3.47 | 3.9 | 1.29 | 0.73 |

Exemplary formulations comprising organic acids and antibiofilm molecules and their antimicrobial results (MIC; relative efficacy) are presented in Table 4 below.

TABLE 4

| Preservative | E. coli | S. aureus | P. aeruginosa | C. albicans | A. brasiliensis |
|---|---|---|---|---|---|
| Sodium | 1875 | 1250 | 2500 | 1875 | 3750 |
| benzoate 58%, | | | | | |
| Potassium | | | | | |
| sorbate 28%, | | | | | |
| Salicylic acid | | | | | |
| 7%, Maltol 7% | | | | | |
| Sodium | 1250 | 1250 | 3750 | 1875 | 3750 |
| benzoate 51%, | | | | | |
| Potassium | | | | | |
| sorbate 21%, | | | | | |
| Salicylic acid | | | | | |
| 28% | | | | | |
| Sodium | 1250 | 1875 | 1250 | 1250 | 2500 |
| Benzoate 58%, | | | | | |
| Potassium | | | | | |
| Sorbate 28%, | | | | | |
| Maltol 14% | | | | | |
| Sodium | 1250 | 1875 | 1250 | 2000 | 5000 |
| Benzoate 86%, | | | | | |
| Maltol 14% | | | | | |

Table 5 presents exemplary formulations comprising zinc species and antibiofilm molecules and their antimicrobial results.

TABLE 5

| Preservative | E. coli | S. aureus | P. aeruginosa | C. albicans | A. brasiliensis |
|---|---|---|---|---|---|
| Salicylic acid | 937 | 937 | 937 | 117 | 156 |
| 10%, Maltol | | | | | |
| 10%, Zinc | | | | | |
| Acetate 80% | | | | | |
| Maltol 10%, | 12.5 | 12.5 | 78 | 0.4 | 4.5 |
| Zinc Acetate | | | | | |
| 86%, Zinc | | | | | |
| Pyrithione 4% | | | | | |
| Salicylic acid | 6.2 | 7.8 | 125 | 0.08 | 2.5 |
| 41.5%, Maltol | | | | | |
| 41.5%, Zinc | | | | | |
| Pyrithione 17% | | | | | |
| Zinc Acetate | 1250 | 625 | 1250 | 130 | 125 |
| 80%, Maltol | | | | | |
| 20% | | | | | |

Table 6 presents exemplary formulations comprising organic acids, zinc species and antibiofilm molecules and their antimicrobial results.

TABLE 6

| Preservative | E. coli | S. aureus | P. aeruginosa | C. albicans | A. brasiliensis |
|---|---|---|---|---|---|
| Sodium | 1250 | 1875 | 1875 | 625 | 937 |
| Benzoate 51%, | | | | | |
| Potassium | | | | | |
| Sorbate 21%, | | | | | |
| Zinc Acetate | | | | | |
| 14%, Salicylic | | | | | |
| acid 7%, Maltol | | | | | |
| 7% | | | | | |
| Benzoic acid | 1875 | 1875 | 2500 | 1250 | 2500 |
| 42%, Sorbic acid | | | | | |
| 20%, Zinc | | | | | |
| Gluconate 10%, | | | | | |
| Maltol 8%, | | | | | |
| Salicylic acid | | | | | |
| 20% | | | | | |
| Sodium | 1250 | 1875 | 1875 | 625 | 937 |
| Benzoate 72%, | | | | | |
| Zinc Acetate | | | | | |
| 14%, Maltol 7%, | | | | | |
| Salicylic acid | | | | | |
| 7% | | | | | |
| Sodium | 937 | 1250 | 1250 | 500 | 312 |
| Benzoate 44%, | | | | | |
| Potassium | | | | | |
| Sorbate 14%, | | | | | |
| Zinc Acetate | | | | | |
| 28%, Maltol | | | | | |
| 14% | | | | | |
| Sodium | 937 | 1250 | 1250 | 1000 | 625 |
| Benzoate 72%, | | | | | |
| Zinc Acetate | | | | | |
| 14%, Maltol | | | | | |
| 14% | | | | | |

### Example 3

### Preservative Effectiveness (Challenge Tests) of Organic Acids and Antibiofilm Agents

The preservative effectiveness was tested to measure the efficiency of the preservatives in different wipes formulations such as nonionic and anionic (Table 1).

Tables 7-9 show efficacy comparison between organic acids (Table 7) and the addition of antibiofilm molecules to organic acids (Tables 8-9).

TABLE 7

| | | | S. | P. | C. | A. |
|---|---|---|---|---|---|---|
| Composition | Time | E. coli | aureus | aeruginosa | albicans | brasiliensis |
| Sodium | Inoculum | 1 × 10\hat{ }6 | 1 × 10\hat{ }6 | 1.1 × 10\hat{ }6 | 1 × 10\hat{ }5 | 9.9 × 10\hat{ }4 |
| benzoate 0.46%, | 2 days | 1 × 10\hat{ }5 | <10 | <10 | 2 × 10\hat{ }4 | 6 × 10\hat{ }3 |
| Potassium | 7 days | 1.1 × 10\hat{ }5 | <10 | <10 | 2.1 × 10\hat{ }4 | 7.1 × 10\hat{ }3 |
| sorbate 0.24%, | 14 days | 1.2 × 10\hat{ }5 | <10 | 8.8 × 10\hat{ }4 | 6.3 × 10\hat{ }3 | 5.8 × 10\hat{ }3 |
| Total 0.7% | 21 days | 2 × 10\hat{ }5 | <10 | 1.6 × 10\hat{ }5 | 5.1 × 10\hat{ }2 | 6.7 × 10\hat{ }2 |
| Nonionic wet | 28 days | 2.1 × 10\hat{ }5 | <10 | 1.8 × 10\hat{ }5 | 7.7 × 10\hat{ }2 | 5 × 10\hat{ }2 |
| wipes (Only | | | | | | |
| organic acids) | | | | | | |
| pH = 5.5 | | | | | | |

TABLE 8

| | | | S. | P. | C. | A. |
|---|---|---|---|---|---|---|
| Composition | Time | E. coli | aureus | aeruginosa | albicans | brasiliensis |
| Sodium | Inoculum | 1.1 × 10\hat{ }6 | 1 × 10\hat{ }6 | 1.2 × 10\hat{ }6 | 1.1 × 10\hat{ }5 | 1 × 10\hat{ }5 |
| benzoate 0.4%, | 2 days | 1 × 10\hat{ }2 | 2.1 × 10\hat{ }2 | <10 | 1.3 × 10\hat{ }4 | 1.2 × 10\hat{ }3 |
| Potassium | 7 days | <10 | <10 | <10 | 7.7 × 10\hat{ }3 | 1.5 × 10\hat{ }3 |
| sorbate 0.2%, | 14 days | <10 | <10 | <10 | 5.6 × 10\hat{ }3 | 5.1 × 10\hat{ }2 |
| Maltol 0.1%, | 21 days | <10 | <10 | <10 | 1 × 10\hat{ }2 | 3 × 10\hat{ }2 |
| Total 0.7% | 28 days | <10 | <10 | <10 | <10 | 6.5 × 10\hat{ }2 |
| Nonionic wet | | | | | | |
| wipes pH = 5.5 | | | | | | |

TABLE 9

| | | | S. | P. | C. | A. |
|---|---|---|---|---|---|---|
| Composition | Time | E. coli | aureus | aeruginosa | albicans | brasiliensis |
| Sodium benzoate | Inoculum | 1.1 × 10\hat{ }6 | 1 × 10\hat{ }6 | 1.1 × 10\hat{ }6 | 1 × 10\hat{ }5 | 1 × 10\hat{ }5 |
| 0.42%, Potassium | 2 days | <10 | <10 | 1.6 × 10\hat{ }2 | 4.1 × 10\hat{ }4 | 1.2 × 10\hat{ }3 |
| sorbate 0.2%, Salicylic | 7 days | <10 | <10 | 2 × 10\hat{ }2 | 1.3 × 10\hat{ }3 | 1 × 10\hat{ }3 |
| acid 0.05%, Maltol | 14 days | 1.5 × 10\hat{ }3 | <10 | 1.8 × 10\hat{ }3 | 1.1 × 10\hat{ }4 | 3.7 × 10\hat{ }2 |
| 0.05%, Total 0.72% | 21 days | 1.2 × 10\hat{ }4 | <10 | 1.9 × 10\hat{ }4 | 1.6 × 10\hat{ }3 | 1.1 × 10\hat{ }2 |
| Nonionic wet wipes | 28 days | 1.1 × 10\hat{ }4 | <10 | 2 × 10\hat{ }4 | <10 | 4 × 10\hat{ }2 |
| pH = 5.5 | | | | | | |

### Example 4

### Preservative Effectiveness (Challenge Tests) of Zinc and Antibiofilm Agents

The antimicrobial preservative effectiveness was tested to measure the efficiency of the preservatives in different wipes formulations such as nonionic and anionic (Table 1).

Tables 10-19 show efficacy comparison between zinc species and antibiofilm molecules. Tables 10-13 show results of the use of zinc acetate and a mixture of salicylic acid and maltol as antibiofilm agent.

TABLE 10

| | | | S. | P. | C. | A. |
|---|---|---|---|---|---|---|
| Composition | Time | E. coli | aureus | aeruginosa | albicans | brasiliensis |
| Salicylic acid | Inoculum | 1 × 10\hat{ }6 | 1 × 10\hat{ }6 | 1.1 × 10\hat{ }6 | 1 × 10\hat{ }5 | 9.9 × 10\hat{ }4 |
| 0.05%, Maltol | 2 days | 1 × 10\hat{ }1 | <10 | <10 | 4 × 10\hat{ }3 | 7 × 10\hat{ }3 |
| 0.05%, Zinc | 7 days | <10 | <10 | <10 | 1.3 × 10\hat{ }4 | 1.2 × 10\hat{ }3 |
| Acetate 0.4%, | 14 days | <10 | <10 | <10 | <10 | 2.5 × 10\hat{ }2 |
| Total 0.5% | 21 days | <10 | <10 | <10 | <10 | 7 × 10\hat{ }2 |
| Nonionic wet | 28 days | <10 | <10 | <10 | <10 | 5.8 × 10\hat{ }2 |
| wipes | | | | | | |
| pH = 5.5 | | | | | | |

TABLE 11

| | | | S. | P. | C. | A. |
|---|---|---|---|---|---|---|
| Composition | Time | E. coli | aureus | aeruginosa | albicans | brasiliensis |
| Salicylic acid | Inoculum | 1.1 × 10\hat{ }6 | 1 × 10\hat{ }6 | 1.2 × 10\hat{ }6 | 1.1 × 10\hat{ }5 | 1 × 10\hat{ }5 |
| 0.05%, Maltol | 2 days | 5 × 10\hat{ }1 | 1 × 10\hat{ }1 | <10 | 7 × 10\hat{ }3 | 1.6 × 10\hat{ }3 |
| 0.05%, Zinc | 7 days | <10 | <10 | <10 | 2.2 × 10\hat{ }2 | 1 × 10\hat{ }3 |
| Acetate 0.4%, | 14 days | <10 | <10 | <10 | <10 | 1.2 × 10\hat{ }3 |
| Total 0.5% | 21 days | <10 | <10 | <10 | <10 | 7.7 × 10\hat{ }2 |
| Nonionic wet | 28 days | <10 | <10 | <10 | <10 | 3.1 × 10\hat{ }2 |
| wipes | | | | | | |
| (Repetition 1) | | | | | | |
| pH = 5.5 | | | | | | |

TABLE 12

| | | | S. | P. | C. | A. |
|---|---|---|---|---|---|---|
| Composition | Time | E. coli | aureus | aeruginosa | albicans | brasiliensis |
| Salicylic acid | Inoculum | 1.1 × 10\hat{ }6 | 1.2 × 10\hat{ }6 | 1 × 10\hat{ }6 | 1.1 × 10\hat{ }5 | 1 × 10\hat{ }5 |
| 0.05%, Maltol | 2 days | <10 | <10 | <10 | 2 × 10\hat{ }3 | 6.3 × 10\hat{ }3 |
| 0.05%, Zinc | 7 days | <10 | <10 | <10 | 1.1 × 10\hat{ }2 | 1 × 10\hat{ }4 |
| Acetate 0.4%, | 14 days | <10 | <10 | <10 | <10 | 2.2 × 10\hat{ }2 |
| Total 0.5% | 21 days | | | | | |
| Nonionic wet | 28 days | | | | | |
| wipes | | | | | | |
| (Repetition 2) | | | | | | |
| pH = 5.5 | | | | | | |

TABLE 13

| | | | S. | P. | C. | A. |
|---|---|---|---|---|---|---|
| Composition | Time | E. coli | aureus | aeruginosa | albicans | brasiliensis |
| Salicylic acid | Inoculum | 1.1 × 10\hat{ }6 | 1.1 × 10\hat{ }6 | 1.2 × 10\hat{ }6 | 1 × 10\hat{ }5 | 1 × 10\hat{ }5 |
| 0.05%, Maltol | 2 days | <10 | 4.5 × 10\hat{ }2 | 6 × 10\hat{ }1 | 1 × 10\hat{ }4 | 1.1 × 10\hat{ }3 |
| 0.05%, Zinc | 7 days | <10 | 7 × 10\hat{ }1 | 1 × 10\hat{ }1 | 3.5 × 10\hat{ }2 | 8.8 × 10\hat{ }2 |
| Acetate 0.4%, | 14 days | <10 | 5 × 10\hat{ }1 | <10 | <10 | 6.3 × 10\hat{ }2 |
| Total 0.5% | 21 days | <10 | 6 × 10\hat{ }1 | <10 | <10 | 5.6 × 10\hat{ }2 |
| Anionic wet | 28 days | <10 | 1 × 10\hat{ }1 | <10 | <10 | 2 × 10\hat{ }2 |
| wipes | | | | | | |
| pH = 5.5 | | | | | | |

Tables 14-16 show results of the use of zinc acetate and maltol as antibiofilm agent.

TABLE 14

| | | | S. | P. | C. | A. |
|---|---|---|---|---|---|---|
| Composition | Time | E. coli | aureus | aeruginosa | albicans | brasiliensis |
| Maltol 0.1%, | Inoculum | 1.1 × 10\hat{ }6 | 1 × 10\hat{ }6 | 1.2 × 10\hat{ }6 | 1.1 × 10\hat{ }5 | 1 × 10\hat{ }5 |
| Zinc Acetate | 2 days | 3 × 10\hat{ }1 | <10 | <10 | 8 × 10\hat{ }3 | 4.4 × 10\hat{ }3 |
| 0.4% | 7 days | <10 | <10 | <10 | 4.2 × 10\hat{ }2 | 1.5 × 10\hat{ }3 |
| Total 0.5% | 14 days | <10 | <10 | <10 | 3 × 10\hat{ }1 | 4.4 × 10\hat{ }3 |
| Nonionic wet | 21 days | <10 | <10 | <10 | 1 × 10\hat{ }1 | 1 × 10\hat{ }3 |
| wipes | 28 days | <10 | <10 | <10 | <10 | 9.6 × 10\hat{ }2 |
| pH = 5.5 | | | | | | |

TABLE 15

| | | | S. | P. | C. | A. |
|---|---|---|---|---|---|---|
| Composition | Time | E. coli | aureus | aeruginosa | albicans | brasiliensis |
| Maltol 0.1%, | Inoculum | 1.3 × 10\hat{ }6 | 1.1 × 10\hat{ }6 | 1 × 10\hat{ }6 | 1.1 × 10\hat{ }5 | 1 × 10\hat{ }5 |
| Zinc Acetate | 2 days | 2.6 × 10\hat{ }2 | <10 | <10 | 1.6 × 10\hat{ }4 | 1.8 × 10\hat{ }4 |
| 0.4% | 7 days | 2 × 10\hat{ }2 | <10 | <10 | 7 × 10\hat{ }3 | 4 × 10\hat{ }2 |
| Total 0.5% | 14 days | | | | | |
| (Repetition) | 21 days | | | | | |
| Nonionic wet | 28 days | | | | | |
| wipes | | | | | | |
| pH = 5.5 | | | | | | |

TABLE 16

| | | | S. | P. | C. | A. |
|---|---|---|---|---|---|---|
| Composition | Time | E. coli | aureus | aeruginosa | albicans | brasiliensis |
| Maltol 0.1%, Zinc | Inoculum | 1.1 × 10\hat{ }6 | 1 × 10\hat{ }6 | 1.2 × 10\hat{ }6 | 1.1 × 10\hat{ }5 | 1 × 10\hat{ }5 |
| Acetate 0.6% | 2 days | <10 | <10 | <10 | 7.8 × 10\hat{ }3 | 5.6 × 10\hat{ }3 |
| Total 0.7% | 7 days | <10 | <10 | <10 | 2.5 × 10\hat{ }2 | 3 × 10\hat{ }3 |
| Nonionic wet | 14 days | <10 | <10 | <10 | 6 × 10\hat{ }1 | 5.8 × 10\hat{ }3 |
| wipes | 21 days | <10 | <10 | <10 | <10 | 1.6 × 10\hat{ }3 |
| pH = 5.5 | 28 days | <10 | <10 | <10 | <10 | 1.1 × 10\hat{ }3 |

Table 17 presents results of the use of zinc acetate, zinc pyrithione and maltol as antibiofilm agent.

TABLE 17

| | | | S. | P. | C. | A. |
|---|---|---|---|---|---|---|
| Composition | Time | E. coli | aureus | aeruginosa | albicans | brasiliensis |
| Maltol 0.05%, | Inoculum | 1 × 10\hat{ }6 | 1 × 10\hat{ }6 | 1.1 × 10\hat{ }6 | 1 × 10\hat{ }5 | 9.9 × 10\hat{ }4 |
| Zinc Acetate | 2 days | <10 | 1.1 × 10\hat{ }5 | <10 | <10 | 1 × 10\hat{ }2 |
| 0.43%, Zinc | 7 days | <10 | 1.5 × 10\hat{ }2 | <10 | <10 | <10 |
| Pyrithione 0.02%, | 14 days | <10 | <10 | <10 | <10 | <10 |
| Total 0.5% | 21 days | <10 | <10 | <10 | <10 | <10 |
| Nonionic wet wipes | 28 days | <10 | <10 | <10 | <10 | <10 |
| pH = 5.5 | | | | | | |

Table 18 shows results of the use of zinc acetate, zinc pyrithione and maltol and salicylic acid as antibiofilm agent.

TABLE 18

| | | | S. | P. | C. | A. |
|---|---|---|---|---|---|---|
| Composition | Time | E. coli | aureus | aeruginosa | albicans | brasiliensis |
| Salicylic acid | Inoculum | 1 × 10\hat{ }6 | 1 × 10\hat{ }6 | 1.1 × 10\hat{ }6 | 1 × 10\hat{ }5 | 9.9 × 10\hat{ }4 |
| 0.05%, Maltol | 2 days | 3.1 × 10\hat{ }4 | 5 × 10\hat{ }2 | 8.1 × 10\hat{ }3 | 3 × 10\hat{ }2 | 4.4 × 10\hat{ }2 |
| 0.05%, Zinc | 7 days | 1.8 × 10\hat{ }3 | 3.5 × 10\hat{ }2 | 1 × 10\hat{ }4 | 1 × 10\hat{ }2 | 3.5 × 10\hat{ }2 |
| Pyrithione | 14 days | 1 × 10\hat{ }2 | 6.3 × 10\hat{ }2 | <10 | <10 | 2 × 10\hat{ }1 |
| 0.02%, Total | 21 days | <10 | <10 | <10 | <10 | <10 |
| 0.12% | 28 days | <10 | <10 | <10 | <10 | <10 |
| Nonionic wet | | | | | | |
| wipes | | | | | | |
| pH = 5.5 | | | | | | |

Table 19 presents the results of the use of zinc gluconate and maltol and salicylic acid as antibiofilm agent.

TABLE 19

| | | | S. | P. | C. | A. |
|---|---|---|---|---|---|---|
| Composition | Time | E. coli | aureus | aeruginosa | albicans | brasiliensis |
| Salicylic acid | Inoculum | 1.1 × 10\hat{ }6 | 1 × 10\hat{ }6 | 1.1 × 10\hat{ }6 | 1 × 10\hat{ }5 | 1 × 10\hat{ }5 |
| 0.05%, Maltol | 2 days | 1.5 × 10\hat{ }3 | <10 | 7.7 × 10\hat{ }5 | 4.8 × 10\hat{ }3 | 1.2 × 10\hat{ }3 |
| 0.05%, Zinc | 7 days | 1 × 10\hat{ }3 | <10 | 1.2 × 10\hat{ }5 | 5.5 × 10\hat{ }2 | 8.9 × 10\hat{ }3 |
| Gluconate 0.8%, | 14 days | 2.1 × 10\hat{ }3 | <10 | 1.5 × 10\hat{ }5 | 2.2 × 10\hat{ }2 | 7.3 × 10\hat{ }3 |
| Total 0.9% | 21 days | 2.1 × 10\hat{ }3 | <10 | 1.5 × 10\hat{ }5 | <10 | 3.9 × 10\hat{ }2 |
| Nonionic wet | 28 days | 2.6 × 10\hat{ }3 | <10 | 1 × 10\hat{ }5 | <10 | 3.8 × 10\hat{ }2 |
| wipes | | | | | | |
| pH = 5.5 | | | | | | |

### Example 5

### Preservative Effectiveness (Challenge Tests) of Organic Acids, Zinc and Antibiofilm Agents

The antimicrobial preservative effectiveness was tested to measure the efficiency of the preservatives in different wipes formulations such as nonionic and anionic (Table 1).

Tables 20-32 show efficacy comparison between different preservative formulations. Tables 20-27 show results of the use of organic acids (sodium benzoate and potassium sorbate), different zinc species and a mixture of salicylic acid and maltol as antibiofilm agent.

TABLE 20

| | | | S. | P. | C. | A. |
|---|---|---|---|---|---|---|
| Composition | Time | E. coli | aureus | aeruginosa | albicans | brasiliensis |
| Sodium | Inoculum | 1 × 10\hat{ }6 | 1.1 × 10\hat{ }6 | 1 × 10\hat{ }6 | 1 × 10\hat{ }5 | 1.1 × 10\hat{ }5 |
| Benzoate 0.35%, | 2 days | <10 | <10 | <10 | 5.7 × 10\hat{ }4 | 7.8 × 10\hat{ }3 |
| Potassium | 7 days | <10 | <10 | <10 | 4.4 × 10\hat{ }2 | 9.6 × 10\hat{ }2 |
| Sorbate 0.15%, | 14 days | <10 | <10 | <10 | 3 × 10\hat{ }1 | 9 × 10\hat{ }2 |
| Zinc Acetate | 21 days | <10 | <10 | <10 | <10 | 5.1 × 10\hat{ }2 |
| 0.1%, Salicylic | 28 days | <10 | <10 | <10 | <10 | 6 × 10\hat{ }1 |
| acid 0.05%, | | | | | | |
| Maltol 0.05%, | | | | | | |
| Total 0.7% | | | | | | |
| Nonionic wet | | | | | | |
| wipes | | | | | | |
| pH = 5.5 | | | | | | |

TABLE 21

| | | | | P. | C. | A. |
|---|---|---|---|---|---|---|
| Composition | Time | E. coli | S. aureus | aeruginosa | albicans | brasiliensis |
| Sodium | Inoculum | 1 × 10\hat{ }6 | 1.1 × 10\hat{ }6 | 1 × 10\hat{ }6 | 1 × 10\hat{ }5 | 1.1 × 10\hat{ }5 |
| Benzoate | 2 days | <10 | <10 | <10 | 1.7 × 10\hat{ }4 | 1.4 × 10\hat{ }4 |
| 0.38%, | 7 days | <10 | <10 | <10 | 1 × 10\hat{ }3 | 1.1 × 10\hat{ }4 |
| Potassium | 14 days | <10 | <10 | <10 | 1.3 × 10\hat{ }3 | 1 × 10\hat{ }4 |
| Sorbate 0.17%, | 21 days | <10 | <10 | <10 | 1.3 × 10\hat{ }3 | 1.1 × 10\hat{ }3 |
| Zinc Acetate | 28 days | <10 | <10 | <10 | 5.2 × 10\hat{ }2 | 1.2 × 10\hat{ }3 |
| 0.1%, Salicylic | | | | | | |
| acid 0.025%, | | | | | | |
| Maltol | | | | | | |
| 0.025%, Total | | | | | | |
| 0.7% | | | | | | |
| Nonionic wet | | | | | | |
| wipes | | | | | | |
| pH = 5.5 | | | | | | |

TABLE 22

| | | | | P. | C. | A. |
|---|---|---|---|---|---|---|
| Composition | Time | E. coli | S. aureus | aeruginosa | albicans | brasiliensis |
| Sodium | Inoculum | 1 × 10\hat{ }6 | 1.1 × 10\hat{ }6 | 1 × 10\hat{ }6 | 1 × 10\hat{ }5 | 1.1 × 10\hat{ }5 |
| Benzoate | 2 days | <10 | 9.4 × 10\hat{ }2 | <10 | 6.1 × 10\hat{ }4 | 7.5 × 10\hat{ }3 |
| 0.38%, | 7 days | <10 | <10 | <10 | 5.8 × 10\hat{ }3 | 8 × 10\hat{ }3 |
| Potassium | 14 days | <10 | <10 | <10 | 3.2 × 10\hat{ }2 | 7.7 × 10\hat{ }3 |
| Sorbate 0.17%, | 21 days | <10 | <10 | <10 | <10 | 7.3 × 10\hat{ }3 |
| Zinc Acetate | 28 days | <10 | <10 | <10 | <10 | 3 × 10\hat{ }2 |
| 0.1%, Maltol | | | | | | |
| 0.05%, Total | | | | | | |
| 0.7% (Without | | | | | | |
| Salicylic acid) | | | | | | |
| Nonionic wet | | | | | | |
| wipes | | | | | | |
| pH = 5.5 | | | | | | |

TABLE 23

| | | | | P. | C. | A. |
|---|---|---|---|---|---|---|
| Composition | Time | E. coli | S. aureus | aeruginosa | albicans | brasiliensis |
| Sodium Benzoate | Inoculum | 1 × 10\hat{ }6 | 1.1 × 10\hat{ }6 | 1 × 10\hat{ }6 | 1 × 10\hat{ }5 | 1.1 × 10\hat{ }5 |
| 0.35%, Potassium | 2 days | <10 | <10 | <10 | 5.7 × 10\hat{ }4 | 7.8 × 10\hat{ }3 |
| Sorbate 0.15%, | 7 days | <10 | <10 | <10 | 4.4 × 10\hat{ }2 | 9.6 × 10\hat{ }2 |
| Zinc Acetate | 14 days | <10 | <10 | <10 | 3 × 10\hat{ }1 | 9 × 10\hat{ }2 |
| 0.1%, Salicylic | 21 days | <10 | <10 | <10 | <10 | 5.1 × 10\hat{ }2 |
| acid 0.05%, | | | | | | |
| Maltol 0.05%, | 28 days | <10 | <10 | <10 | <10 | 6 × 10\hat{ }1 |
| Total 0.7% | | | | | | |
| Nonionic wet | | | | | | |
| wipes | | | | | | |
| pH = 5.5 | | | | | | |

TABLE 24

| | | | | P. | C. | A. |
|---|---|---|---|---|---|---|
| Composition | Time | E. coli | S. aureus | aeruginosa | albicans | brasiliensis |
| Benzoic acid | Inoculum | 1.2 × 10\hat{ }6 | 1.1 × 10\hat{ }6 | 1.3 × 10\hat{ }6 | 1 × 10\hat{ }5 | 1 × 10\hat{ }5 |
| 0.42%, Sorbic | 2 days | 1 × 10\hat{ }2 | <10 | 1.1 × 10\hat{ }2 | 9.5 × 10\hat{ }3 | 1 × 10\hat{ }4 |
| acid 0.2%, Zinc | 7 days | <10 | <10 | <10 | 1 × 10\hat{ }2 | 9.6 × 10\hat{ }3 |
| Gluconate | 14 days | <10 | <10 | <10 | 3.5 × 10\hat{ }2 | 1 × 10\hat{ }3 |
| 0.1%, Maltol | 21 days | <10 | <10 | <10 | 3 × 10\hat{ }2 | 1.1 × 10\hat{ }3 |
| 0.08%, Salicylic | 28 days | <10 | <10 | <10 | <10 | 8.6 × 10\hat{ }2 |
| acid 0.2%, Total | | | | | | |
| 1% | | | | | | |
| Nonionic wet | | | | | | |
| wipes pH = 5.5 | | | | | | |

TABLE 25

| | | | | P. | C. | A. |
|---|---|---|---|---|---|---|
| Composition | Time | E. coli | S. aureus | aeruginosa | albicans | brasiliensis |
| Sodium | Inoculum | 1 × 10\hat{ }6 | 1.1 × 10\hat{ }6 | 1 × 10\hat{ }6 | 1 × 10\hat{ }5 | 1.1 × 10\hat{ }5 |
| Benzoate 0.42%, | 2 days | <10 | <10 | <10 | 5.5 × 10\hat{ }2 | <10 |
| Potassium | 7 days | 2.5 × 10\hat{ }2 | <10 | <10 | 5 × 10\hat{ }1 | <10 |
| Sorbate 0.21%, | 14 days | 1.2 × 10\hat{ }3 | <10 | <10 | <10 | <10 |
| Zinc Pyrithion | 21 days | 2.2 × 10\hat{ }3 | <10 | <10 | <10 | <10 |
| 0.02%, Maltol | 28 days | 2.6 × 10\hat{ }3 | <10 | <10 | <10 | <10 |
| 0.025%, | | | | | | |
| Salicylic acid | | | | | | |
| 0.025%, Total | | | | | | |
| 0.7% | | | | | | |
| Nonionic wet | | | | | | |
| wipes pH = 5.5 | | | | | | |

TABLE 26

| | | | | P. | C. | A. |
|---|---|---|---|---|---|---|
| Composition | Time | E. coli | S. aureus | aeruginosa | albicans | brasiliensis |
| Sodium Benzoate | Inoculum | 1 × 10\hat{ }6 | 1.1 × 10\hat{ }6 | 1 × 10\hat{ }6 | 1 × 10\hat{ }5 | 1.1 × 10\hat{ }5 |
| 0.32%, Potassium | 2 days | <10 | <10 | <10 | 7.7 × 10\hat{ }4 | 1.1 × 10\hat{ }4 |
| Sorbate 0.13%, | 7 days | <10 | <10 | <10 | 6.5 × 10\hat{ }2 | 1 × 10\hat{ }4 |
| Zinc Acetate | 14 days | <10 | <10 | <10 | 8.2 × 10\hat{ }2 | 4.5 × 10\hat{ }3 |
| 0.2%, Salicylic | 21 days | <10 | <10 | <10 | 3.8 × 10\hat{ }2 | 1.1 × 10\hat{ }3 |
| acid 0.025%, | | | | | | |
| Maltol 0.025%, | 28 days | <10 | <10 | <10 | 1 × 10\hat{ }3 | 1.6 × 10\hat{ }3 |
| Total 0.7% | | | | | | |
| Nonionic wet | | | | | | |
| wipes | | | | | | |
| pH = 5.5 | | | | | | |

TABLE 27

| | | | | P. | C. | A. |
|---|---|---|---|---|---|---|
| Composition | Time | E. coli | S. aureus | aeruginosa | albicans | brasiliensis |
| Sodium Benzoate | Inoculum | 1 × 10\hat{ }6 | 1.1 × 10\hat{ }6 | 1 × 10\hat{ }6 | 1 × 10\hat{ }5 | 1.1 × 10\hat{ }5 |
| 0.43%, Potassium | 2 days | 5 × 10\hat{ }1 | <10 | <10 | 8.5 × 10\hat{ }2 | 5.5 × 10\hat{ }4 |
| Sorbate 0.215%, | 7 days | <10 | <10 | <10 | 6 × 10\hat{ }1 | 5.1 × 10\hat{ }4 |
| Zinc Pyrithion | 14 days | <10 | <10 | <10 | 2 × 10\hat{ }1 | 8.6 × 10\hat{ }3 |
| 0.005%, Salicylic | 21 days | <10 | <10 | <10 | <10 | 5.9 × 10\hat{ }3 |
| acid 0.05%, Total | 28 days | <10 | <10 | <10 | <10 | 1 × 10\hat{ }2 |
| 0.7% | | | | | | |
| Nonionic wet | | | | | | |
| wipes pH = 5.5 | | | | | | |

Tables 28-30 present the results of the use of preservatives free of salicylic acid. The level of use of zinc acetate was increased and maltol used as antibiofilm agent.

TABLE 28

| | | | S. | P. | C. | A. |
|---|---|---|---|---|---|---|
| Composition | Time | E. coli | aureus | aeruginosa | albicans | brasiliensis |
| Sodium Benzoate | Inoculum | 1.1 × 10\hat{ }6 | 1 × 10\hat{ }6 | 1.2 × 10\hat{ }6 | 1 × 10\hat{ }5 | 1.1 × 10\hat{ }5 |
| 0.35%, Potassium | 2 days | <10 | <10 | <10 | 1.1 × 10\hat{ }4 | 6.5 × 10\hat{ }2 |
| Sorbate 0.15%, | 7 days | <10 | <10 | <10 | 1 × 10\hat{ }4 | 1 × 10\hat{ }4 |
| Zinc Acetate | 14 days | <10 | <10 | <10 | 2.6 × 10\hat{ }2 | 1 × 10\hat{ }4 |
| 0.1%, Maltol | 21 days | <10 | <10 | 1 × 10\hat{ }2 | 3 × 10\hat{ }1 | 9.8 × 10\hat{ }3 |
| 0.1%, Total 0.7%, | 28 days | <10 | <10 | 6.1 × 10\hat{ }2 | <10 | 8.6 × 10\hat{ }2 |
| Nonionic wet | | | | | | |
| wipes | | | | | | |
| pH = 5.5 | | | | | | |

TABLE 29

| | | | S. | P. | C. | A. |
|---|---|---|---|---|---|---|
| Composition | Time | E. coli | aureus | aeruginosa | albicans | brasiliensis |
| Sodium Benzoate | Inoculum | 1.1 × 10\hat{ }6 | 1 × 10\hat{ }6 | 1.2 × 10\hat{ }6 | 1 × 10\hat{ }5 | 1.1 × 10\hat{ }5 |
| 0.33%, Potassium | 2 days | <10 | <10 | <10 | 1.2 × 10\hat{ }4 | 1.6 × 10\hat{ }4 |
| Sorbate 0.12%, | 7 days | <10 | <10 | <10 | 6.6 × 10\hat{ }2 | 1.3 × 10\hat{ }4 |
| Zinc Acetate | 14 days | <10 | <10 | <10 | <10 | 1.1 × 10\hat{ }3 |
| 0.15%, Maltol | 21 days | <10 | <10 | <10 | <10 | 1.4 × 10\hat{ }3 |
| 0.1%, Total 0.7%, | 28 days | <10 | <10 | <10 | 1 × 10\hat{ }2 | 8 × 10\hat{ }1 |
| Nonionic wet | | | | | | |
| wipes | | | | | | |
| pH = 5.5 | | | | | | |

The best result was obtained for a level of use of zinc acetate of 0.2% (Table 30).

TABLE 30

| | | | | P. | C. | A. |
|---|---|---|---|---|---|---|
| Composition | Time | E. coli | S. aureus | aeruginosa | albicans | brasiliensis |
| Sodium Benzoate | Inoculum | 1.1 × 10\hat{ }6 | 1 × 10\hat{ }6 | 1.2 × 10\hat{ }6 | 1 × 10\hat{ }5 | 1.1 × 10\hat{ }5 |
| 0.3%, Potassium | 2 days | <10 | 3.6 × 10\hat{ }2 | 1.5 × 10\hat{ }4 | 9.7 × 10\hat{ }3 | 5.5 × 10\hat{ }3 |
| Sorbate 0.1%, | 7 days | <10 | 1 × 10\hat{ }2 | 1.1 × 10\hat{ }2 | 3.3 × 10\hat{ }2 | 1 × 10\hat{ }3 |
| Zinc Acetate | 14 days | <10 | <10 | <10 | <10 | 1.6 × 10\hat{ }3 |
| 0.2%, Maltol | 21 days | <10 | <10 | <10 | <10 | 1.5 × 10\hat{ }3 |
| 0.1%, Total 0.7% | 28 days | <10 | <10 | <10 | <10 | 7.8 × 10\hat{ }2 |
| Nonionic wet | | | | | | |
| wipes | | | | | | |
| pH = 5.5 | | | | | | |

The use of salicylic acid-free preservative was compared in nonionic vs. anionic formulations (Tables 31-32).

TABLE 31

| | | | | P. | C. | A. |
|---|---|---|---|---|---|---|
| Composition | Time | E. coli | S. aureus | aeruginosa | albicans | brasiliensis |
| Sodium | Inoculum | 1 × 10\hat{ }6 | 1.1 × 10\hat{ }6 | 1 × 10\hat{ }6 | 1 × 10\hat{ }5 | 1.1 × 10\hat{ }5 |
| Benzoate | 2 days | <10 | 9.4 × 10\hat{ }2 | <10 | 6.1 × 10\hat{ }4 | 7.5 × 10\hat{ }3 |
| 0.38%, | 7 days | <10 | <10 | <10 | 5.8 × 10\hat{ }3 | 8 × 10\hat{ }3 |
| Potassium | 14 days | <10 | <10 | <10 | 3.2 × 10\hat{ }2 | 7.7 × 10\hat{ }3 |
| Sorbate 0.17%, | 21 days | <10 | <10 | <10 | <10 | 7.3 × 10\hat{ }3 |
| Zinc Acetate | 28 days | <10 | <10 | <10 | <10 | 3 × 10\hat{ }2 |
| 0.1%, Maltol | | | | | | |
| 0.05%, Total | | | | | | |
| 0.7%, Nonionic | | | | | | |
| wet wipes | | | | | | |
| pH = 5.5 | | | | | | |

TABLE 32

| | | | | P. | C. | A. |
|---|---|---|---|---|---|---|
| Composition | Time | E. coli | S. aureus | aeruginosa | albicans | brasiliensis |
| Sodium | Inoculum | 1.1 × 10\hat{ }6 | 1.1 × 10\hat{ }6 | 1.2 × 10\hat{ }6 | 1 × 10\hat{ }5 | 1 × 10\hat{ }5 |
| Benzoate | 2 days | 6 × 10\hat{ }1 | 5.3 × 10\hat{ }3 | <10 | 1.3 × 10\hat{ }4 | 6 × 10\hat{ }3 |
| 0.38%, | 7 days | 2 × 10\hat{ }1 | 5 × 10\hat{ }1 | <10 | 9.7 × 10\hat{ }2 | 5.8 × 10\hat{ }3 |
| Potassium | 14 days | <10 | <10 | <10 | <10 | 1.5 × 10\hat{ }3 |
| Sorbate | 21 days | <10 | <10 | <10 | <10 | 6.7 × 10\hat{ }3 |
| 0.17%, Zinc | 28 days | <10 | <10 | <10 | <10 | 1.2 × 10\hat{ }3 |
| Acetate 0.1%, | | | | | | |
| Maltol | | | | | | |
| 0.05%, | | | | | | |
| Total 0.7%, | | | | | | |
| Anionic wet | | | | | | |
| wipes | | | | | | |
| pH = 5.5 | | | | | | |

Tables 33-34 present preservative formulations with increased level of use and potassium sorbate free and salicylic acid free.

TABLE 33

| | | | | P. | C. | A. |
|---|---|---|---|---|---|---|
| Composition | Time | E. coli | S. aureus | aeruginosa | albicans | brasiliensis |
| Sodium | Inoculum | 1.3 × 10\hat{ }6 | 1.1 × 10\hat{ }6 | 1 × 10\hat{ }6 | 1.1 × 10\hat{ }5 | 1 × 10\hat{ }5 |
| Benzoate | 2 days | <10 | <10 | <10 | 1.5 × 10\hat{ }4 | 1.3 × 10\hat{ }4 |
| 0.52%, Zinc | 7 days | <10 | <10 | <10 | 1.1 × 10\hat{ }3 | 2.6 × 10\hat{ }2 |
| Acetate 0.1%, | 14 days | | | | | |
| Maltol 0.1% | 21 days | | | | | |
| Total 0.72% | 28 days | | | | | |
| Nonionic wet | | | | | | |
| wipes | | | | | | |
| pH = 5.5 | | | | | | |

TABLE 34

| | | | | P. | C. | A. |
|---|---|---|---|---|---|---|
| Composition | Time | E. coli | S. aureus | aeruginosa | albicans | brasiliensis |
| Sodium Benzoate 0.8%, | Inoculum | 1.3 × 10\hat{ }6 | 1.1 × 10\hat{ }6 | 1 × 10\hat{ }6 | 1.1 × 10\hat{ }5 | 1 × 10\hat{ }5 |
| Zinc Acetate 0.1%, | 2 days | <10 | <10 | <10 | 1.5 × 10\hat{ }3 | 1.2 × 10\hat{ }3 |
| Maltol 0.1% | 7 days | <10 | <10 | <10 | 3.7 × 10\hat{ }2 | 1 × 10\hat{ }1 |
| Total 1% | 14 days | | | | | |
| Nonionic wet wipes | 21 days | | | | | |
| pH = 5.5 | 28 days | | | | | |

### Example 6

### Anti Microbial Activity Evaluation and Preservative Effectiveness (Challenge Tests) of Organic Acids and Zinc

Exemplary formulations comprising organic acids and zinc compounds and their antimicrobial results (MIC; relative efficacy) are presented in Table 35 below.

TABLE 35

| Preservative | E. coli | S. aureus | P. aeruginosa | C. albicans | A. brasiliensis |
|---|---|---|---|---|---|
| Sodium | 2800 | 2800 | 4000 | 2000 | 2000 |
| Benzoate 58% + | | | | | |
| Potassium | | | | | |
| Sorbate 28% + | | | | | |
| Zinc Gluconate | | | | | |
| 14% pH = 5.5 | | | | | |
| Sodium | 1500 | 1750 | 2000 | 1000 | 1500 |
| Benzoate 58% + | | | | | |
| Potassium | | | | | |
| Sorbate 28% + | | | | | |
| Zinc Gluconate | | | | | |
| 14% pH = 4.5 | | | | | |
| Sodium | 937 | 1250 | 1250 | 625 | 500 |
| Benzoate 58% + | | | | | |
| Potassium | | | | | |
| Sorbate 28% + | | | | | |
| Zinc Acetate | | | | | |
| 14% pH = 5.5 | | | | | |
| Sodium | 937 | 1250 | 1250 | 1000 | 625 |
| Benzoate 86% + | | | | | |
| Zinc Acetate | | | | | |
| 14% pH = 5.5 | | | | | |

The wet wipes formulations used are shown (previously in Table 1) and in Table 36.

TABLE 36

| Cationic | | Zwitterionic | | Surfactant free | |
|---|---|---|---|---|---|
| Water | q.s. | Water | q.s. | Water | q.s. |
| Chamomile | 0.05% | Chamomile | 0.05% | Chamomile | 0.05% |
| extract | | extract | | extract | |
| Aloe Vera | 0.001% | Aloe Vera | 0.001% | Aloe Vera | 0.001% |
| extract | | extract | | extract | |
| D-Panthenol | 0.05% | D-Panthenol | 0.05% | D-Panthenol | 0.05% |
| Glycerin | 0.5% | Glycerin | 0.5% | Glycerin | 0.5% |
| Citric acid | q.s. | Citric acid | q.s | Citric acid | q.s. |
| Polysorbate 20 | 0.6% | Polysorbate 20 | 0.6% | Preservative | 0.5-1% |
| (surfactant) | | | | | |
| Guar | 0.2% | Cocamidopropyl | 0.2% | pH | 5.5 |
| hydroxypropyltrimonium | | betain | | | |
| chloride | | | | | |
| (surfactant) | | | | | |
| Preservative | 0.5-1% | Preservative | 0.5-1% | | |

| | | |
|---|---|---|
| pH 5.5 | pH | 5.5 |

Tables 37-52 show results of the use of organic acids and different zinc species in different wet wipes formulations.

TABLE 37

| | | | | P. | C. | A. |
|---|---|---|---|---|---|---|
| Composition | Time | E. coli | S. aureus | aeruginosa | albicans | brasiliensis |
| Benzoic acid 0.42%, | Inoculum | 1.2 × 10\hat{ }6 | 1.1 × 10\hat{ }6 | 1.3 × 10\hat{ }6 | 1 × 10\hat{ }5 | 1 × 10\hat{ }5 |
| Sorbic acid 0.2%, Zinc | 2 days | 1.1 × 10\hat{ }4 | <10 | 3.5 × 10\hat{ }4 | 3 × 10\hat{ }4 | 1.1 × 10\hat{ }4 |
| Gluconate 0.1%, level | 7 days | <10 | <10 | <10 | 6.3 × 10\hat{ }3 | 8.6 × 10\hat{ }3 |
| of use 0.72% | 14 days | <10 | <10 | <10 | 7.1 × 10\hat{ }2 | 9.5 × 10\hat{ }2 |
| Nonionic wet wipes | 21 days | <10 | <10 | <10 | <10 | 7.7 × 10\hat{ }2 |
| pH = 5.5 | 28 days | <10 | <10 | <10 | <10 | 2.6 × 10\hat{ }2 |

TABLE 38

| | | | | P. | C. | A. |
|---|---|---|---|---|---|---|
| Composition | Time | E. coli | S. aureus | aeruginosa | albicans | brasiliensis |
| Benzoic acid 0.29%, | Inoculum | 1.1 × 10\hat{ }6 | 1.2 × 10\hat{ }6 | 1. × 10\hat{ }6 | 1 × 10\hat{ }5 | 9.9 × 10\hat{ }4 |
| Sorbic acid 0.14%, | 2 days | <10 | <10 | 1 × 10\hat{ }5 | 7.8 × 10\hat{ }4 | 8 × 10\hat{ }3 |
| Zinc Gluconate 0.07%, | 7 days | <10 | <10 | <10 | 8.6 × 10\hat{ }3 | 4 × 10\hat{ }4 |
| level of use 0.5% | 14 days | <10 | <10 | <10 | 3 × 10\hat{ }1 | 4.3 × 10\hat{ }4 |
| Nonionic wet wipes | 21 days | <10 | <10 | <10 | 3 × 10\hat{ }1 | 3.5 × 10\hat{ }4 |
| pH = 5.5 | 28 days | <10 | <10 | 1 × 10\hat{ }3 | <10 | 8.7 × 10\hat{ }3 |

TABLE 39

| | | | | P. | C. | A. |
|---|---|---|---|---|---|---|
| Composition | Time | E. coli | S. aureus | aeruginosa | albicans | brasiliensis |
| Benzoic acid 0.29%, | Inoculum | 1.1 × 10\hat{ }6 | 1.2 × 10\hat{ }6 | 1. × 10\hat{ }6 | 1 × 10\hat{ }5 | 9.9 × 10\hat{ }4 |
| Sorbic acid 0.14%, Zinc | 2 days | <10 | <10 | 1.1 × 10\hat{ }5 | 1.6 × 10\hat{ }2 | 3.8 × 10\hat{ }3 |
| Gluconate 0.07%, level | 7 days | <10 | <10 | 1.8 × 10\hat{ }5 | <10 | 3.7 × 10\hat{ }3 |
| of use 0.5% | 14 days | <10 | <10 | <10 | <10 | 4 × 10\hat{ }4 |
| Nonionic wet wipes | 21 days | <10 | <10 | <10 | <10 | 4 × 10\hat{ }1 |
| pH = 4.5 | 28 days | <10 | <10 | <10 | <10 | 9 × 10\hat{ }1 |

TABLE 40

| | | | | P. | C. | A. |
|---|---|---|---|---|---|---|
| Composition | Time | E. coli | S. aureus | aeruginosa | albicans | brasiliensis |
| Sodium benzoate | Inoculum | 1 × 10\hat{ }6 | 1 × 10\hat{ }6 | 1.1 × 10\hat{ }6 | 1 × 10\hat{ }5 | 9.9 × 10\hat{ }4 |
| 0.42%, Potassium | 2 days | <10 | <10 | <10 | 8.8 × 10\hat{ }3 | 7 × 10\hat{ }3 |
| sorbate 0.2%, Zinc | 7 days | <10 | <10 | <10 | 8.6 × 10\hat{ }3 | 5.5 × 10\hat{ }4 |
| Acetate 0.1%, Total | 14 days | <10 | <10 | <10 | <10 | 3 × 10\hat{ }4 |
| 0.72% | | | | | | |
| Nonionic wet wipes | 21 days | <10 | <10 | <10 | <10 | 6.3 × 10\hat{ }3 |
| pH = 5.5 | 28 days | <10 | <10 | <10 | 1.5 × 10\hat{ }2 | 4 × 10\hat{ }2 |

TABLE 41

| | | | | P. | C. | A. |
|---|---|---|---|---|---|---|
| Composition | Time | E. coli | S. aureus | aeruginosa | albicans | brasiliensis |
| Sodium benzoate | Inoculum | 1.2 × 10\hat{ }6 | 1 × 10\hat{ }6 | 1.1 × 10\hat{ }6 | 1.1 × 10\hat{ }5 | 1 × 10\hat{ }5 |
| 0.62%, Zinc Acetate | 2 days | 1.8 × 10\hat{ }3 | <10 | <10 | 8 × 10\hat{ }3 | 1.2 × 10\hat{ }3 |
| 0.1%, Total 0.72% | 7 days | <10 | <10 | <10 | 2.1 × 10\hat{ }2 | 2.5 × 10\hat{ }3 |
| Nonionic wet wipes | 14 days | <10 | <10 | <10 | 5 × 10\hat{ }1 | 2 × 10\hat{ }3 |
| (Potassium sorbate | 21 days | <10 | <10 | <10 | <10 | 9.6 × 10\hat{ }2 |
| free) pH = 5.5 | 28 days | <10 | <10 | <10 | <10 | 1 × 10\hat{ }3 |

TABLE 42

| | | | | P. | C. | A. |
|---|---|---|---|---|---|---|
| Composition | Time | E. coli | S. aureus | aeruginosa | albicans | brasiliensis |
| Sodium benzoate | Inoculum | 1.1 × 10\hat{ }6 | 1.2 × 10\hat{ }6 | 1 × 10\hat{ }6 | 1.1 × 10\hat{ }5 | 1 × 10\hat{ }5 |
| 0.62%, Zinc Acetate | 2 days | 6.3 × 10\hat{ }2 | <10 | 2.5 × 10\hat{ }2 | 1.5 × 10\hat{ }3 | 1.6 × 10\hat{ }3 |
| 0.1%, Total 0.72% | 7 days | <10 | <10 | <10 | <10 | 1.5 × 10\hat{ }3 |
| Nonionic wet wipes | 14 days | | | | | |
| (Repetition) | 21 days | | | | | |
| pH = 5.5 | 28 days | | | | | |

TABLE 43

| | | | | P. | C. | A. |
|---|---|---|---|---|---|---|
| Composition | Time | E. coli | S. aureus | aeruginosa | albicans | brasiliensis |
| Sodium benzoate | Inoculum | 1.2 × 10\hat{ }6 | 1 × 10\hat{ }6 | 1.1 × 10\hat{ }6 | 1.1 × 10\hat{ }5 | 1 × 10\hat{ }5 |
| 0.62%, Zinc Acetate | 2 days | 1.8 × 10\hat{ }3 | <10 | <10 | 8 × 10\hat{ }3 | 1.2 × 10\hat{ }3 |
| 0.1%, Total 0.72% | 7 days | <10 | <10 | <10 | 2.1 × 10\hat{ }2 | 2.5 × 10\hat{ }3 |
| Nonionic wet wipes | 14 days | <10 | <10 | <10 | 5 × 10\hat{ }1 | 2 × 10\hat{ }3 |
| pH = 5.5 | 21 days | <10 | <10 | <10 | <10 | 9.6 × 10\hat{ }2 |
| | 28 days | <10 | <10 | <10 | <10 | 1 × 10\hat{ }3 |

TABLE 44

| | | | | P. | C. | A. |
|---|---|---|---|---|---|---|
| Composition | Time | E. coli | S. aureus | aeruginosa | albicans | brasiliensis |
| Sodium benzoate | Inoculum | 1 × 10\hat{ }6 | 1 × 10\hat{ }6 | 1.1 × 10\hat{ }6 | 1 × 10\hat{ }5 | 9.9 × 10\hat{ }4 |
| 0.42%, Potassium | 2 days | <10 | <10 | <10 | 4 × 10\hat{ }2 | 3.5 × 10\hat{ }2 |
| sorbate 0.2%, Zinc | 7 days | <10 | <10 | <10 | 3.2 × 10\hat{ }2 | 5.8 × 10\hat{ }3 |
| Pyrithion 0.02%, Total | 14 days | <10 | <10 | <10 | <10 | 6 × 10\hat{ }3 |
| 0.64% | 21 days | <10 | <10 | <10 | <10 | 3.3 × 10\hat{ }4 |
| Nonionic wet wipes | 28 days | <10 | <10 | <10 | <10 | 5.2 × 10\hat{ }3 |
| pH = 5.5 | | | | | | |

TABLE 45

| | | | | P. | C. | A. |
|---|---|---|---|---|---|---|
| Composition | Time | E. coli | S. aureus | aeruginosa | albicans | brasiliensis |
| Sodium benzoate | Inoculum | 1.1 × 10\hat{ }6 | 1.1 × 10\hat{ }6 | 1.2 × 10\hat{ }6 | 1 × 10\hat{ }5 | 1 × 10\hat{ }5 |
| 0.42%, Potassium | 2 days | <10 | 6.3 × 10\hat{ }2 | <10 | 1 × 10\hat{ }2 | <10 |
| sorbate 0.2%, Zinc | 7 days | <10 | 2 × 10\hat{ }1 | <10 | 8 × 10\hat{ }1 | <10 |
| Pyrithion 0.02%, Total | 14 days | <10 | <10 | <10 | <10 | <10 |
| 0.64% | | | | | | |
| Anionic wet wipes | 21 days | <10 | <10 | <10 | <10 | <10 |
| pH = 5.5 | 28 days | <10 | <10 | <10 | <10 | <10 |

TABLE 46

| | | | | P. | C. | A. |
|---|---|---|---|---|---|---|
| Composition | Time | E. coli | S. aureus | aeruginosa | albicans | brasiliensis |
| Sodium benzoate | Inoculum | 1.2 × 10\hat{ }6 | 1 × 10\hat{ }6 | 1.1 × 10\hat{ }6 | 1.1 × 10\hat{ }5 | 1 × 10\hat{ }5 |
| 0.42%, Potassium | 2 days | 3 × 10\hat{ }3 | 5 × 10\hat{ }1 | <10 | 5.6 × 10\hat{ }3 | 1.3 × 10\hat{ }3 |
| sorbate 0.2%, Zinc | 7 days | 1.5 × 10\hat{ }3 | <10 | <10 | 2.2 × 10\hat{ }2 | 2 × 10\hat{ }3 |
| Acetate 0.1%, Total | 14 days | <10 | <10 | <10 | <10 | 1.5 × 10\hat{ }3 |
| 0.72% | 21 days | <10 | <10 | <10 | <10 | 1.6 × 10\hat{ }3 |
| Nonionic wet wipes | 28 days | <10 | <10 | <10 | <10 | 5.2 × 10\hat{ }2 |
| (Repetition) pH = 5.5 | | | | | | |

TABLE 47

| | | | | P. | C. | A. |
|---|---|---|---|---|---|---|
| Composition | Time | E. coli | S. aureus | aeruginosa | albicans | brasiliensis |
| Sodium benzoate | Inoculum | 1.2 × 10\hat{ }6 | 1 × 10\hat{ }6 | 1.1 × 10\hat{ }6 | 1.1 × 10\hat{ }5 | 1 × 10\hat{ }5 |
| 0.42%, Potassium | | | | | | |
| sorbate 0.2%, Zinc | 2 days | <10 | 1.5 × 10\hat{ }3 | 2.3 × 10\hat{ }4 | 1 × 10\hat{ }4 | 1.2 × 10\hat{ }3 |
| Acetate 0.1%, Total | | | | | | |
| 0.72% | 7 days | <10 | 1 × 10\hat{ }3 | <10 | 5.2 × 10\hat{ }2 | 9.6 × 10\hat{ }2 |
| Surfactant free wet | 14 days | <10 | <10 | <10 | 1.6 × 10\hat{ }4 | 1.7 × 10\hat{ }3 |
| wipes | 21 days | <10 | <10 | <10 | 1.7 × 10\hat{ }4 | 2.1 × 10\hat{ }3 |
| pH = 5.5 | 28 days | <10 | <10 | <10 | 1.5 × 10\hat{ }4 | 1.6 × 10\hat{ }3 |

TABLE 48

| | | | | P. | C. | A. |
|---|---|---|---|---|---|---|
| Composition | Time | E. coli | S. aureus | aeruginosa | albicans | brasiliensis |
| Sodium benzoate | Inoculum | 1.2 × 10\hat{ }6 | 1 × 10\hat{ }6 | 1.1 × 10\hat{ }6 | 1.1 × 10\hat{ }5 | 1 × 10\hat{ }5 |
| 0.42%, Potassium | 2 days | <10 | <10 | <10 | 8.3 × 10\hat{ }3 | 1.1 × 10\hat{ }4 |
| sorbate 0.2%, Zinc | 7 days | <10 | <10 | <10 | 9.5 × 10\hat{ }3 | 1 × 10\hat{ }4 |
| Acetate 0.1%, Total | 14 days | <10 | <10 | <10 | 1.1 × 10\hat{ }4 | 1 × 10\hat{ }4 |
| 0.72% | 21 days | <10 | <10 | <10 | 1.2 × 10\hat{ }4 | 1.8 × 10\hat{ }3 |
| Cationic wet wipes | 28 days | <10 | <10 | <10 | 1 × 10\hat{ }4 | 1.7 × 10\hat{ }3 |
| pH = 5.5 | | | | | | |

TABLE 49

| | | | | P. | C. | A. |
|---|---|---|---|---|---|---|
| Composition | Time | E. coli | S. aureus | aeruginosa | albicans | brasiliensis |
| Sodium benzoate | Inoculum | 1.2 × 10\hat{ }6 | 1 × 10\hat{ }6 | 1.1 × 10\hat{ }6 | 1.1 × 10\hat{ }5 | 1 × 10\hat{ }5 |
| 0.42%, Potassium | | | | | | |
| sorbate 0.2%, Zinc | 2 days | <10 | <10 | <10 | 1.6 × 10\hat{ }3 | 7.5 × 10\hat{ }3 |
| Acetate 0.1%, Total | 7 days | <10 | <10 | <10 | 3.3 × 10\hat{ }2 | 1.2 × 10\hat{ }4 |
| 0.72% | 14 days | <10 | <10 | <10 | 8.8 × 10\hat{ }3 | 1.1 × 10\hat{ }4 |
| Zwitterionic wet wipes | 21 days | <10 | <10 | <10 | 2.6 × 10\hat{ }3 | 1 × 10\hat{ }4 |
| pH = 5.5 | 28 days | <10 | <10 | <10 | 2 × 10\hat{ }3 | 9.8 × 10\hat{ }2 |

Tables 50-52 show results of preservatives comprising organic acids and zinc acetate with increased level of use.

TABLE 50

| | | | | P. | C. | A. |
|---|---|---|---|---|---|---|
| Composition | Time | E. coli | S. aureus | aeruginosa | albicans | brasiliensis |
| Sodium benzoate | Inoculum | 1.1 × 10\hat{ }6 | 1 × 10\hat{ }6 | 1.2 × 10\hat{ }6 | 1.1 × 10\hat{ }5 | 1 × 10\hat{ }5 |
| 0.42%, Potassium | 2 days | <10 | <10 | <10 | 1.1 × 10\hat{ }4 | 8.8 × 10\hat{ }2 |
| sorbate 0.2%, Zinc | 7 days | <10 | <10 | <10 | 1 × 10\hat{ }4 | 5.5 × 10\hat{ }3 |
| Acetate 0.1%, Total | 14 days | <10 | <10 | <10 | 7.5 × 10\hat{ }3 | 8.6 × 10\hat{ }3 |
| 0.72% (Repetition) | 21 days | <10 | <10 | <10 | 5 × 10\hat{ }2 | 5.2 × 10\hat{ }3 |
| Nonionic wet wipes | 28 days | <10 | <10 | <10 | <10 | 1.3 × 10\hat{ }3 |
| pH = 5.5 | | | | | | |

TABLE 51

| | | | | P. | C. | A. |
|---|---|---|---|---|---|---|
| Composition | Time | E. coli | S. aureus | aeruginosa | albicans | brasiliensis |
| Sodium benzoate | Inoculum | 1 × 10\hat{ }6 | 1 × 10\hat{ }6 | 1.2 × 10\hat{ }6 | 1.1 × 10\hat{ }5 | 1 × 10\hat{ }5 |
| 0.37%, Potassium | | | | | | |
| sorbate 0.15%, Zinc | 2 days | <10 | <10 | 1.5 × 10\hat{ }3 | 1 × 10\hat{ }4 | 5.2 × 10\hat{ }3 |
| Acetate 0.2%, Total | 7 days | <10 | <10 | <10 | 8.6 × 10\hat{ }3 | 5 × 10\hat{ }3 |
| 0.72% | 14 days | <10 | <10 | <10 | 3.3 × 10\hat{ }2 | 1.2 × 10\hat{ }3 |
| Nonionic wet wipes | 21 days | <10 | <10 | <10 | 2.5 × 10\hat{ }2 | 1 × 10\hat{ }3 |
| pH = 5.5 | 28 days | <10 | <10 | <10 | <10 | 1 × 10\hat{ }3 |

TABLE 52

| | | | | P. | C. | A. |
|---|---|---|---|---|---|---|
| Composition | Time | E. coli | S. aureus | aeruginosa | albicans | brasiliensis |
| Sodium benzoate | Inoculum | 1.2 × 10\hat{ }6 | 1 × 10\hat{ }6 | 1.1 × 10\hat{ }6 | 1.1 × 10\hat{ }5 | 1 × 10\hat{ }5 |
| 0.54%, Potassium | | | | | | |
| sorbate 0.26%, Zinc | 2 days | <10 | <10 | <10 | 1.1 × 10\hat{ }3 | 6.1 × 10\hat{ }3 |
| Acetate 0.2%, Total | 7 days | <10 | <10 | <10 | 1.3 × 10\hat{ }3 | 6.5 × 10\hat{ }2 |
| 1% | 14 days | <10 | <10 | <10 | <10 | 4.8 × 10\hat{ }2 |
| Nonionic wet wipes | 21 days | <10 | <10 | <10 | <10 | 5.2 × 10\hat{ }2 |
| pH = 5.5 | 28 days | <10 | <10 | <10 | <10 | 5.5 × 10\hat{ }2 |

The inventors further tested additional antimicrobial compositions comprising the active ingredients (also referred to herein as “preservative” or “preservative combination”) as follows:
1. About 0.5-0.6% w/w of the organic acid (e.g. benzoic acid and/or a salt thereof), about 0.1-0.5% w/w zinc agent (e.g. a zinc (II) salt, such as zinc acetate) and about 0.1-0.3% w/w an antibiofilm agent (e.g. maltol).
2. About 0.5-1.2% w/w of organic acids (e.g. benzoic acid, sorbic acid and/or salts thereof), and about 0.05-0.15% w/w of an antibiofilm agent (e.g. maltol).
3. About 0.5-0.6% w/w of the organic acid (e.g. benzoic acid and/or a salt thereof) and about 0.1-0.5% w/w zinc agent (e.g. a zinc (II) salt, such as zinc acetate).

The weight ratio of the active ingredients (or preservative) within the tested antimicrobial compositions was between about 0.7-1.5%. It should be appreciated that lower or higher amounts of the active ingredients can be implemented, dependent on a particular application. In some embodiments, a weight ratio of the active ingredients within the antimicrobial composition of the invention is as described herein (e.g. between about 0.5 and 5%, or between about 0.5 and 2%).

The inventors tested the preservatives in 2-3 different aqueous formulations: cationic, anionic, and nonionic formulation respectively. The exact composition of these formulations are described hereinabove (e.g. in Tables 1 and 36).

The tested antimicrobial compositions exhibited significant antimicrobial activity, resulting almost complete eradication (or at least 2-4 orders of magnitude reduction of the initial CFU) of microbes including bacteria and fungi from the inoculated wet textile substrate including PET, viscose, wood pulp and combinations thereof (e.g. in a form of wet wipes). The antimicrobial activity has been evaluated at pH 5.5, and 4.5. The tested antimicrobial compositions exhibited significant antimicrobial activity for a time period of about 30 days, about 60 days or more. Long-term antimicrobial tests are currently ongoing. It is presumed that the antimicrobial compositions may eradicate or substantially inhibit microbial load for a time period of up to 1 year, up to 2 years, or any range between when stored under appropriate storage conditions, such as described herein.

Furthermore, the inventors successfully tested the above mentioned preservatives in (i) mixed microorganisms tests (inoculum comprising different bacteria species, and/or different fungi/mold species), (ii) at successive inoculation (e.g. after 2, 7 and 28 days)

Furthermore, the inventors obtained similar results e.g. almost complete eradication (or at least 2-4 orders of magnitude reduction of the initial CFU) of microbes including bacteria and fungi from the inoculated wet wipes using additional zinc (II) salts instead of zinc acetate, such as zinc lactate and zinc glycinate. The tested zinc salts have been applied at similar concentrations as the above disclosed formulations (e.g. about 0.4-0.5% w/w).

Moreover, based on solid experimental results, the inventors presume similar antibiofilm activity for compounds such as D-Tyrosine, Naringin, Deferiprone, Mannose, Urea, Acetyl Cysteine, Diacetyl Cysteine, Gallic acid, Ethylexylglycerin, Ethylacetoacetate, Hordenine, Tyrosol, Hydroxytyrosol, Tyramine, Lactobionic Acid, Anthranilic acid, Trigonelline, Caffeine, Tryptophan, including any salt, any stereoisomer, any tautomer and/or any antimicrobially active derivate thereof. Accordingly, it is presumed that the above mentioned compounds will exhibit synergistic antimicrobial activity similar to the tested antibiofilm compounds such as maltol. Thus, it is presumed that the above mentioned compounds can be successfully implemented within the antimicrobial compositions of the invention at a minimum antimicrobial effective concentration of about 0.05-0.2, or about 0.1-0.2% w/w. Additionally, it is postulated that Chlorogenic acid, Levulinic acid, Methyl anthranilate may exhibit similar synergistic activity (e.g. as an antibiofilm, agent) and thus can be successfully implemented within the antimicrobial compositions of the invention.

Of note, based on the experimental results it was concluded that the exemplary antimicrobial compositions of the invention (e.g. preservative combinations 1-3, described hereinabove) showed a substantial synergistic effect, by substantially increasing (e.g. 1-6 orders of magnitude) antimicrobial activity, compared to single treatments with any one of the constituents.

Similar antimicrobial affect has been observed by introducing the antimicrobial compositions of the invention (e.g. preservative combinations 1-3, described hereinabove) into additional cosmeceutical articles, such as cream and shampoo.

The composition of an exemplary cream formulation and an exemplary shampoo formulation are disclosed in Table 53 below.

TABLE 53

| Nonionic- Leave on basic cream | | Anionic-Rinse off shampoo | |
|---|---|---|---|
| Water | q.s. | Water | q.s. |
| Glycerin | 4% | Glycerin | 0.3% |
| Cetearyl Glucoside | 5% | Cocamidopropyl | 1% |
| (and) Cetearyl | | Betaine | |
| alcohol | | | |
| Caprylic Capric | 3% | Cocamide DEA | 1% |
| Triglycerides | | | |
| Cetearyl Alcohol | 4.8% | SLES 70% | 15% |
| (and) Ceteareth-20 | | | |
| Tocopheryl acetate | 0.1% | Lactic acid | q.s. |
| Preservative | 0.5-1% | NaCl | 3.5% |
| Lactic acid | q.s. | Preservative | 0.5-1.2% |
| pH | 5.5 | pH | 5.5 |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An antimicrobial composition comprising (i) an organic acid, a salt thereof or both; or (ii) an antibiofilm agent; and further comprising (iii) a zinc compound; wherein:
a ratio between the (i) and the (iii) is 10:1 and 4:1 by weight, respectively;
a ratio between the (ii) and the (iii) is between 2:1 and 1:5 by weight, respectively;
a weight per weight concentration of the (i) within said antimicrobial composition is between 15% and 95%; and
wherein a weight per weight concentration of the (iii) within said antimicrobial composition is between 10% and 25%

2. The antimicrobial composition of claim 1, wherein a weight per weight concentration of the (i) within said antimicrobial composition is between 30% and 90%; and wherein a weight per weight concentration of the (ii) within said antimicrobial composition is between 5% and 25%.

3. (canceled)

4. (canceled)

5. The antimicrobial composition of claim 1 comprising between 0.5% and 95% (w/w) of said zinc compound; optionally wherein said zinc compound comprises a zinc(II) cation including any salt, any complex thereof, or both; further optionally wherein said zinc compound comprises zinc acetate, zinc pyrithione, zinc gluconate, zinc lactate, zinc glycinate or any combination thereof.

6. (canceled)

7. (canceled)

8. An antimicrobial composition comprising an antibiofilm agent and an organic acid, wherein a w/w ratio between the antibiofilm agent and the organic acid within said antimicrobial composition is between 1:20 and 1:3.

9. The antimicrobial composition of claim 8, wherein the organic acid comprises a first acid and a second acid, and wherein a w/w ratio between the first acid and the second acid within the antimicrobial composition is between 1:3 and 3:1; and wherein a w/w ratio between the antibiofilm agent and the organic acid within said antimicrobial composition is between 1:10 and 1:8; optionally wherein said antibiofilm agent comprises any one of a pyrone, salicylic acid D-Tyrosine, Naringin, Deferiprone, Mannose, Urea, Acetyl Cysteine, Diacetyl Cysteine, Gallic acid, Ethylexylglycerin, Ethylacetoacetate, Hordenine, Tyrosol, Hydroxytyrosol, Tyramine, Lactobionic Acid, Levulinic acid, Anthranilic acid, Methyl anthranilate, Trigonelline, Caffeine, Chlorogenic acid, Tryptophan including any salt thereof, any ester thereof, any derivative thereof, and any combination thereof; optionally wherein said pyrone is selected from the group consisting of maltol, ethyl maltol or any derivative thereof.

10. (canceled)

11. (canceled)

12. The antimicrobial composition of claim 1, wherein said antibiofilm agent comprises any one of a pyrone, salicylic acid D-Tyrosine, Naringin, Deferiprone, Mannose, Urea, Acetyl Cysteine, Diacetyl Cysteine, Gallic acid, Ethylexylglycerin, Ethylacetoacetate, Hordenine, Tyrosol, Hydroxytyrosol, Tyramine, Lactobionic Acid, Levulinic acid, Anthranilic acid, Methyl anthranilate, Trigonelline, Caffeine, Chlorogenic acid, Tryptophan including any salt thereof, any ester thereof, any derivative thereof, and any combination thereof; optionally wherein said pyrone is selected from the group consisting of maltol, ethyl maltol or any derivative thereof.

13. (canceled)

14. The antimicrobial composition of claim 1, comprising between 1% and 80% (w/w) of said antibiofilm agent and optionally comprising two antibiofilm agents in about a 1:1 (w/w) ratio.

15. (canceled)

16. The antimicrobial composition of claim 1, wherein said organic acid comprises between 1 and 7 carbon atoms; optionally wherein said organic acid comprises C1-C7 carboxylic acid; optionally wherein said organic acid is selected from the group consisting of: benzoic acid, dehydroacetic acid, sorbic acid, salicylic acid, lactic acid, citric acid, and any combination thereof.

17. (canceled)

18. (canceled)

19. An article, comprising an antimicrobial effective amount of the composition of claim 1, and an acceptable carrier; wherein the antimicrobial effective amount is less than 2% w/w; and wherein said acceptable carrier is selected from a cosmeceutically acceptable carrier, a pharmaceutically acceptable carrier and a food acceptable carrier.

20. (canceled)

21. (canceled)

22. (canceled)

23. The article of claim 19, wherein the article is characterized by a substantially reduced pathogen load, compared to a similar article devoid of said antimicrobial composition; and wherein said substantially reduced comprises at least 10 times reduced pathogen load.

24. (canceled)

25. The article of claim 23, wherein said substantially reduced pathogen load is for a period ranging between 1 week and 2 years.

26. The article of claim 19, being a cosmeceutical product, a personal care product, or both.

27. The article of claim 26, wherein said personal care product is selected from a fabric, a bandage, a wipe, a pledget, a swab, a suppository, a dressing, a solution, a mousse, a pad, and a patch, or any combination thereof.

28. The article of claim 26, wherein said cosmeceutical product is in a form of a formulation selected from the group consisting of: a liquid, a solution, a paste, a cream, a lotion, a foam, a gel, an emulsion, an ointment, and a soap.

29. The article of claim 19, being for use in the treatment of a condition selected from medical, cosmetic and cosmeceutical condition.

30.-40. (canceled)
